SEQUENCE LISTING (27 sequences)

SEQ ID NO: 1 - protein, 824 aa, Homo sapiens
MRGLGLWLLGAMMLPAIAPSRPWALMEQYEVVLPWRLPGPRVRRALPSHLGLHPERVSYVLGATGHNFTLHLRKNRDLLGSGYTETYTAANGSEVTEQPRGQDHCFYQGHVEGYPDSAASLSTCAGLRGFFQVGSDLHLIEPLDEGGEGGRHAVYQAEHLLQTAGTCGVSDDSLGSLLGPRTAAVFRPRPGDSLPSRETRYVELYVVVDNAEFQMLGSEAAVRHRVLEVVNHVDKLYQKLNFRVVLVGLEIWNSQDRFHVSPDPSVTLENLLTWQARQRTRRHLHDNVQLITGVDFTGTTVGFARVSAMCSHSSGAVNQDHSKNPVGVACTMAHEMGHNLGMDHDENVQGCRCQERFEAGRCIMAGSIGSSFPRMFSDCSQAYLESFLERPQSVCLANAPDLSHLVGGPVCGNLFVERGEQCDCGPPEDCRNRCCNSTTCQLAEGAQCAHGTCCQECKVKPAGELCRPKKDMCDLEEFCDGRHPECPEDAFQENGTPCSGGYCYNGACPTLAQQCQAFWGPGGQAAEESCFSYDILPGCKASRYRADMCGVLQCKGGQQPLGRAICIVDVCHALTTEDGTAYEPVPEGTRCGPEKVCWKGRCQDLHVYRSSNCSAQCHNHGVCNHKQECHCHAGWAPPHCAKLLTEVHAASGSLPVFVVVVLVLLAVVLVTLAGIIVYRKARSRILSRNVAPKTTMGRSNPLFHQAASRVPAKGGAPAPSRGPQELVPTTHPGQPARHPASSVALKRPPPAPPVTVSSPPFPVPVYTRQAPKQVIKPTFAPPVPPVKPGAGAANPGPAEGAVGPKVALKPPIQRKQGAGAPTAP

SEQ ID NO: 2 - protein, 181 aa, artificial sequence
  Synthetic peptide
MIAPSRPWALMEQYEVVLPWRLPGPRVRRALPSHLGLHPERVSYVLGATGHNFTLHLRKNRDLLGSGYTETYTAANGSEVTEQPRGQDHCFYQGHVEGYPDSAASLSTCAGLRGFFQVGSDLHLIEPLDEGGEGGRHAVYQAEHLLQTAGTCGVSDDSLGSLLGPRTAAVFRPRPGDSLPS

SEQ ID NO: 3 - protein, 181 aa, Artificial Sequence
  Synthetic peptide
  misc_feature (20)..(20): Xaa can be any naturally occurring amino acid
  misc_feature (26)..(26): Xaa can be any naturally occurring amino acid
  misc_feature (28)..(29): Xaa can be any naturally occurring amino acid
  misc_feature (90)..(90): Xaa can be any naturally occurring amino acid
  misc_feature (109)..(109): Xaa can be any naturally occurring amino acid
  misc_feature (137)..(149): Xaa can be any naturally occurring amino acid
  misc_feature (152)..(152): Xaa can be any naturally occurring amino acid
MIAPSRPWALMEQYEVVLPXRLPGPXVXXALPSHLGLHPERVSYVLGATGHNFTLHLRKNRDLLGSGYTETYTAANGSEVTEQPRGQDHXFYQGHVEGYPDSAASLSTXAGLRGFFQVGSDLHLIEPLDEGGEGGRXXXXXXXXXXXXXGTXGVSDDSLGSLLGPRTAAVFRPRPGDSLPS

SEQ ID NO: 4 - protein, 181 aa, artificial sequence
  synthetic peptide
MIAPSRPWALMEQYEVVLPRRLPGPRVRAALPSHLGLHPERVSYVLGATGHNFTLHLRKNRDLLGSGYTETYTAANGSEVTEQPRGQDHSFYQGHVEGYPDSAASLSTSAGLRGFFQVGSDLHLIEPLDEGGEGGRHAVYQAEHLLQTAGTSGVSDDSLGSLLGPRTAAVFRPRPGDSLPS

SEQ ID NO: 5 - protein, 180 aa, artificial sequence
  synthetic peptide
MIAPSRPWALMEQYEVVLPWRLPGPKVRKLPSHLGLHPERVSYVLGATGHNFTLHLRKNRDLLGSGYTETYTAANGSEVTEQPRGQDHSFYQGHVEGYPDSAASLSTSAGLRGFFQVGSDLHLIEPLDEGGEGGRHAVYQAEHLLQTAGTSGVSDDSLGSLLGPRTAAVFRPRPGDSLPS

SEQ ID NO: 6 - protein, 181 aa, Artificial Sequence
  Synthetic peptide
MIAPSRPWALMEQYEVVLPWRLPGPKVKKALPSHLGLHPERVSYVLGATGHNFTLHLRKNRDLLGSGYTETYTAANGSEVTEQPRGQDHSFYQGHVEGYPDSAASLSTSAGLRGFFQVGSDLHLIEPLDEGGEGGRHAVYQAEHLLQTAGTSGVSDDSLGSLLGPRTAAVFRPRPGDSLPS

SEQ ID NO: 7 - protein, 181 aa, Artificial Sequence
  Synthetic peptide
MIAPSRPWALMEQYEVVLPWRLPGPKVKKALPSHLGLHPERVSYVLGATGHNFTLHLRKNRDLLGSGYTETYTAANGSEVTEQPRGQDHSFYQGHVEGYPDSAASLSTSAGLRGFFQVGSDLHLIEPLDEGGEGGRHAVYAAAHLLATAGTSGVSDDSLGSLLGPRTAAVFRPRPGDSLPS

SEQ ID NO: 8 - protein, 181 aa, Artificial Sequence
  Synthetic peptide
MIAPSRPWALMEQYEVVLPWRLPGPKVKKALPSHLGLHPERVSYVLGATGHNFTLHLRKNRDLLGSGYTETYTAANGSEVTEQPRGQDHSFYQGHVEGYPDSAASLSTSAGLRGFFQVGSDLHLIEPLDEGGEGGRHAVYNANHLLNTAGTSGVSDDSLGSLLGPRTAAVFRPRPGDSLPS

SEQ ID NO: 9 - protein, 181 aa, Artificial Sequence
  Synthetic peptide
MIAPSRPWALMEQYEVVLPRRLPGPRVRAALPSHLGLHPERVSYVLGATGHNFTLHLRKNRDLLGSGYTETYTAANGSEVTEQPRGQDHSFYQGHVEGYPDSAASLSTSAGLRGFFQVGSDLHLIEPLDEGGEGGRHAVYNANHLLNTAGTSGVSDDSLGSLLGPRTAAVFRPRPGDSLPS

SEQ ID NO: 10 - protein, 5 aa, artificial sequence
  Synthetic peptide
GSCGS

SEQ ID NO: 11 - protein, 5 aa, Artificial Sequence
  Synthetic peptide
GSGSC

SEQ ID NO: 12 - protein, 824 aa, Homo sapiens [sequence omitted]

SEQ ID NO: 13 - protein, 824 aa, homo sapiens
  misc_feature (35)..(35): Xaa can be any naturally occurring amino acid
MRGLGLWLLGAMMLPAIAPSRPWALMEQYEVVLPXRLPGPRVRRALPSHLGLHPERVSYVLGATGHNFTLHLRKNRDLLGSGYTETYTAANGSEVTEQPRGQDHCFYQGHVEGYPDSAASLSTCAGLRGFFQVGSDLHLIEPLDEGGEGGRHAVYQAEHLLQTAGTCGVSDDSLGSLLGPRTAAVFRPRPGDSLPSRETRYVELYVVVDNAEFQMLGSEAAVRHRVLEVVNHVDKLYQKLNFRVVLVGLEIWNSQDRFHVSPDPSVTLENLLTWQARQRTRRHLHDNVQLITGVDFTGTTVGFARVSAMCSHSSGAVNQDHSKNPVGVACTMAHEMGHNLGMDHDENVQGCRCQERFEAGRCIMAGSIGSSFPRMFSDCSQAYLESFLERPQSVCLANAPDLSHLVGGPVCGNLFVERGEQCDCGPPEDCRNRCCNSTTCQLAEGAQCAHGTCCQECKVKPAGELCRPKKDMCDLEEFCDGRHPECPEDAFQENGTPCSGGYCYNGACPTLAQQCQAFWGPGGQAAEESCFSYDILPGCKASRYRADMCGVLQCKGGQQPLGRAICIVDVCHALTTEDGTAYEPVPEGTRCGPEKVCWKGRCQDLHVYRSSNCSAQCHNHGVCNHKQECHCHAGWAPPHCAKLLTEVHAASGSLPVFVVVVLVLLAVVLVTLAGIIVYRKARSRILSRNVAPKTTMGRSNPLFHQAASRVPAKGGAPAPSRGPQELVPTTHPGQPARHPASSVALKRPPPAPPVTVSSPPFPVPVYTRQAPKQVIKPTFAPPVPPVKPGAGAANPGPAEGAVGPKVALKPPIQRKQGAGAPTAP

SEQ ID NO: 14 - protein, 181 aa, artificial sequence
  synthetic peptide
MIAPSRPWALMEQYEVVLPRRLPGPRVRRALPSHLGLHPERVSYVLGATGHNFTLHLRKNRDLLGSGYTETYTAANGSEVTEQPRGQDHCFYQGHVEGYPDSAASLSTCAGLRGFFQVGSDLHLIEPLDEGGEGGRHAVYQAEHLLQTAGTCGVSDDSLGSLLGPRTAAVFRPRPGDSLPS

SEQ ID NO: 15 - protein, 181 aa, artificial sequence
  synthetic peptide
MIAPSRPWALMEQYEVVLPRRLPGPRVRGALPSHLGLHPERVSYVLGATGHNFTLHLRKNRDLLGSGYTETYTAANGSEVTEQPRGQDHSFYQGHVEGYPDSAASLSTSAGLRGFFQVGSDLHLIEPLDEGGEGGRHAVYQAEHLLQTAGTSGVSDDSLGSLLGPRTAAVFRPRPGDSLPS

SEQ ID NO: 16 - protein, 181 aa, artificial sequence
  synthetic peptide
MIAPSRPWALMEQYEVVLPRRLPGPRVRRALPSHLGLHPERVSYVLGATGHNFTLHLRKNRDLLGSGYTETYTAANGSEVTEQPRGQDHCLYQGHVEGYPDSAASLSTCAGLRGFFQVGSDLHLIEPLDEGGEGGRHAVYQAEHLLQTAGTCGVSDDSLGSLLGPRTAAVFRPRPGDSLPS

SEQ ID NO: 17 - protein, 181 aa, artificial sequence
  synthetic peptide
MIAPSRPWALMEQYEVVLPWRLPGPKVRKALPSHLGLHPERVSYVLGATGHNFTLHLRKNRDLLGSGYTETYTAANGSEVTEQPRGQDHSFYQGHVEGYPDSAASLSTSAGLRGFFQVGSDLHLIEPLDEGGEGGRHAVYQAEHLLQTAGTSGVSDDSLGSLLGPRTAAVFRPRPGDSLPS

SEQ ID NO: 18 - protein, 181 aa, artificial sequence
  synthetic peptide
  misc_feature (20)..(20): Xaa can be any naturally occurring amino acid
  misc_feature (26)..(26): Xaa can be any naturally occurring amino acid
  misc_feature (28)..(29): Xaa can be any naturally occurring amino acid
  misc_feature (58)..(59): Xaa can be any naturally occurring amino acid
  misc_feature (61)..(61): Xaa can be any naturally occurring amino acid
  misc_feature (90)..(90): Xaa can be any naturally occurring amino acid
  misc_feature (109)..(109): Xaa can be any naturally occurring amino acid
  misc_feature (152)..(152): Xaa can be any naturally occurring amino acid
MIAPSRPWALMEQYEVVLPXRLPGPXVXXALPSHLGLHPERVSYVLGATGHNFTLHLXXNXDLLGSGYTETYTAANGSEVTEQPRGQDHXFYQGHVEGYPDSAASLSTXAGLRGFFQVGSDLHLIEPLDEGGEGGRHAVYQAEHLLQTAGTXGVSDDSLGSLLGPRTAAVFRPRPGDSLPS

SEQ ID NO: 19 - protein, 181 aa, artificial sequence
  synthetic peptide
  misc_feature (20)..(20): Xaa can be any naturally occurring amino acid
  misc_feature (26)..(26): Xaa can be any naturally occurring amino acid
  misc_feature (28)..(29): Xaa can be any naturally occurring amino acid
  misc_feature (58)..(59): Xaa can be any naturally occurring amino acid
  misc_feature (61)..(61): Xaa can be any naturally occurring amino acid
  misc_feature (90)..(90): Xaa can be any naturally occurring amino acid
  misc_feature (109)..(109): Xaa can be any naturally occurring amino acid
  misc_feature (137)..(149): Xaa can be any naturally occurring amino acid
  misc_feature (152)..(152): Xaa can be any naturally occurring amino acid
MIAPSRPWALMEQYEVVLPXRLPGPXVXXALPSHLGLHPERVSYVLGATGHNFTLHLXXNXDLLGSGYTETYTAANGSEVTEQPRGQDHXFYQGHVEGYPDSAASLSTXAGLRGFFQVGSDLHLIEPLDEGGEGGRXXXXXXXXXXXXXGTXGVSDDSLGSLLGPRTAAVFRPRPGDSLPS

SEQ ID NO: 20 - protein, 182 aa, artificial sequence
  synthetic peptide
  misc_feature (20)..(20): Xaa can be any naturally occurring amino acid
  misc_feature (26)..(26): Xaa can be any naturally occurring amino acid
  misc_feature (28)..(29): Xaa can be any naturally occurring amino acid
  misc_feature (53)..(53): Xaa can be any naturally occurring amino acid
  misc_feature (59)..(60): Xaa can be any naturally occurring amino acid
  misc_feature (62)..(62): Xaa can be any naturally occurring amino acid
  misc_feature (77)..(77): Xaa can be any naturally occurring amino acid
  misc_feature (91)..(91): Xaa can be any naturally occurring amino acid
  misc_feature (110)..(110): Xaa can be any naturally occurring amino acid
  misc_feature (138)..(150): Xaa can be any naturally occurring amino acid
  misc_feature (153)..(153): Xaa can be any naturally occurring amino acid
MIAPSRPWALMEQYEVVLPXRLPGPXVXXALPSHLGLHPERVSYVLGATGHNXFTLHLXXNXDLLGSGYTETYTAAXGSEVTEQPRGQDHXFYQGHVEGYPDSAASLSTXAGLRGFFQVGSDLHLIEPLDEGGEGGRXXXXXXXXXXXXXGTXGVSDDSLGSLLGPRTAAVFRPRPGDSLPS

SEQ ID NO: 21 - DNA, 576 bp, Homo sapiens
atgattgcccccagccggccctgggccctcatggagcagtatgaggtcgtgttgccgcggcgtctgccaggcccccgagtccgccgagctctgccctcccacttgggcctgcacccagagagggtgagctacgtccttggggccacagggcacaacttcaccctccacctgcggaagaacagggacctgctgggttccggctacacagagacctatacggctgccaatggctccgaggtgacggagcagcctcgcgggcaggaccactgcttataccagggccacgtagaggggtacccggactcagccgccagcctcagcacctgtgccggcctcaggggtttcttccaggtggggtcagacctgcacctgatcgagcccctggatgaaggtggcgagggcggacggcacgccgtgtaccaggctgagcacctgctgcagacggccgggacctgcggggtcagcgacgacagcctgggcagcctcctgggaccccggacggcagccgtcttcaggcctcggcccggggactctctgccatccggatctggatctcatcatcatcatcatcattaa

SEQ ID NO: 22 - DNA, 576 bp, artificial sequence
  synthetic peptide
atgattgcccccagccggccctgggccctcatggagcagtatgaggtcgtgttgccgcggcgtctgccaggcccccgagtccgcgctgctctgccctcccacttgggcctgcacccagagagggtgagctacgtccttggggccacagggcacaacttcaccctccacctgcggaagaacagggacctgctgggttccggctacacagagacctatacggctgccaatggctccgaggtgacggagcagcctcgcgggcaggaccactctttctaccagggccacgtagaggggtacccggactcagccgccagcctcagcacctctgccggcctcaggggtttcttccaggtggggtcagacctgcacctgatcgagcccctggatgaaggtggcgagggcggacggcacgccgtgtaccaggctgagcacctgctgcagacggccgggacctctggggtcagcgacgacagcctgggcagcctcctgggaccccggacggcagccgtcttcaggcctcggcccggggactctctgccatccggatctggatctcatcatcatcatcatcattaa

SEQ ID NO: 23 - protein, 591 aa, artificial sequence
  synthetic peptide
ATGGGCTCGTGTGGCTCCATTGCGCCGAGCCGTCCGTGGGCGCTGATGGAACAGTATGAAGTGGTGTTACCATGGCGGCTGCCCGGTCCGAAAGTGCGCAAAGCGCTGCCGTCGCATCTGGGTCTGCACCCTGAGCGTGTGAGCTATGTGCTGGGCGCGACCGGCCATAACTTCACATTGCATCTTCGTAAAAATCGTGATTTACTGGGCTCCGGCTACACCGAAACCTATACGGCCGCTAATGGTAGCGAAGTGACCGAACAGCCGCGTGGCCAGGATCATAGCTTTTACCAGGGCCATGTGGAAGGGTATCCTGATAGCGCGGCAAGCCTGAGCACATCGGCTGGCCTGCGGGGCTTTTTTCAGGTGGGAAGCGACTTGCACCTGATTGAACCGCTGGATGAAGGCGGCGAAGGTGGGCGTCATGCGGTGTATCAGGCGGAACATCTGTTGCAGACAGCGGGCACAAGCGGCGTCAGCGACGATTCCCTTGGCAGCCTTCTGGGACCACGTACCGCGGCAGTTTTTCGTCCGCGTCCGGGCGACAGCCTGCCTAGCGGCTCTGGGAGCCATCACCACCATCATCACTAA

SEQ ID NO: 24 - DNA, 576 bp, artificial sequence
  synthetic peptide
atgatagcgccaagccgtccctgggcgctgatggaacagtatgaagtggtgctgccgtggcgtctgccaggtccacgtgtgcgtcgtgcgttgccgtctcatctgggcctgcaccctgaacgtgtgagctatgtgttaggcgcgaccggccataatttcaccctgcatcttcgtaaaaatcgtgatctgctgggcagcggctataccgagacctacacagcggccaatggcagcgaagtgaccgaacagccgcgtggccaagatcattgcttttatcagggccatgtggaaggatacccggattcagcggcgagcctgagcacttgcgccggtttacggggcttttttcaggtggggagcgatttgcatctgattgaaccgctggatgaaggaggggagggaggccgccatgcggtgtatcaagcggagcatcttttacaaaccgcgggaacctgcggcgtgagcgatgacagcctggggtctttgttgggaccacgtaccgcagcggtctttcgtccgcgtccgggcgatagcttaccgtccggctcggggtcccatcatcaccaccatcattga

SEQ ID NO: 25 - DNA, 579 bp, artificial sequence
  synthetic peptide
atgatcgcgccgtctcgtccgtgggcgctgatggaacagtatgaagtggtgctgccccgtcgtctgccgggtccccgtgtgcgtggcgccctgccgagccatctgggcctgcatccggaacgtgtgagctatgtgctgggtgcgaccggccacaattttaccctgcacctgcgtaaaaatcgtgatctgctgggctcaggatataccgaaacctatacagcggccaacggctcagaggtgaccgaacagccgcgtggccaggaccatagcttctatcagggccatgtggaaggttaccccgactcagcagcgagcctgagcacaagcgcgggattgcgtggattttttcaggtgggcagcgatctgcatctgattgaaccgctggatgaagggggcgaaggaggccgtcatgcggtgtatcaggcggaacacttgctgcagaccgcgggtacctctggcgtcagcgatgacagcctgggtagcctgttgggaccgcgtaccgcggcagtatttcgtccgcgtccgggcgatagccttccgagcggtagctgcggctcccatcatcaccaccatcattaa

SEQ ID NO: 26 - DNA, 576 bp, artificial sequence
  synthetic peptide
atgattgccccgagccgtccgtgggcgctgatggaacagtatgaagtggtgctgccgcgtcgtctgccgggaccgcgtgtgcgtcgtgcgctgccgagccatctgggcctgcatccggaacgtgtgagctatgtgcttggcgcgaccggccataatttcacgttgcatctgcgtaaaaatcgtgatctgctgggcagcggctataccgaaacctacacagctgccaatggcagcgaagtgaccgaacagccgcgtggccaggatcattgcttttatcagggccatgtggaggggtatccagatagtgccgcgagcctgagcacctgtgcgggcttgcgtggcttttttcaggtggggagcgatctgcatcttattgaaccgctggatgaagggggcgaaggtggccgtcatgccgtgtatcaagcggaacatttactgcagaccgcgggtacctgcggcgtgagcgatgattccctgggtagcctgttggggcctcgtaccgctgcggtgtttcgtccgcgtccgggcgatagtctgcctagcggtagcgggtcgcatcatcaccaccatcactaa

SEQ ID NO: 27 - DNA, 540 bp, homo sapiens
attgcccccagccggccctgggccctcatggagcagtatgaggtcgtgttgccgtggcgtctgccaggcccccgagtccgccgagctctgccctcccacttgggcctgcacccagagagggtgagctacgtccttggggccacagggcacaacttcaccctccacctgcggaagaacagggacctgctgggctccggctacacagagacctatacggctgccaatggctccgaggtgacggagcagcctcgcgggcaggaccactgcttctaccagggccacgtagaggggtacccggactcagccgccagcctcagcacctgtgccggcctcaggggtttcttccaggtggggtcagacctgcacctgatcgagcccctggatgaaggtggcgagggcggacggcacgccgtgtaccaggctgagcacctgctgcagacggccgggacctgcggggtcagcgacgacagcctgggcagcctcctgggaccccggacggcagccgtcttcaggcctcggcccggggactctctgccatcc

(19) United States
(12) Patent Application Publication
(10) Pub. No.: US 2022/0213169 A1
(43) Pub. Date: Jul. 7, 2022

(54) METHODS AND COMPOSITIONS FOR INHIBITING ADAM8 BIOLOGICAL ACTIVITIES

(71) Applicant: Verra Therapeutics, LLC, Ithaca, NY (US)

(72) Inventors: Marcia L. Moss, Apex, NC (US); Robert Rasmussen, Lansing, NY (US); Christopher Prince, Ithaca, NY (US)

(21) Appl. No.: 17/610,016

(22) PCT Filed: May 11, 2020
(86) PCT No.: PCT/US2020/032370

§ 371 Date: Nov. 9, 2021

Related U.S. Application Data
(60) Provisional application No. 62/845,544, filed on May 9, 2019.

(51) Int. Cl. C07K 14/705; C12N 9/64

(52) U.S. Cl. CPC C07K 14/70596; C07K 2319/21; C12N 9/6489

(57) ABSTRACT

This disclosure provides ADAM8 modulating peptides, nucleic acids encoding ADAM8 modulating peptides, and methods for using the same to modulate ADAM8 biological activities in vitro and/or in vivo, to inhibit ADAM8 biological activities associated with diseases or disorders in subjects including gene therapy or cell based therapies. Specifically, methods are provided to decrease inflammation, and to inhibit undesirable cellular proliferation, fibrosis, and angiogenesis. In some embodiments, the ADAM8 modulating peptides or nucleic acids encoding them include modifications of one or more amino acids of the human ADAM8 prodomain amino acid sequence, and in some embodiments the ADAM8 modulating peptides include other modifications such as but not limited to the addition of PEG groups.

### CROSS REFERENCE TO RELATED APPLICATIONS

This application claims the benefit of U.S. Provisional No. 62/845,544 filed May 9, 2019, inventors Marcia L. Moss et al., entitled “METHODS AND COMPOSITIONS FOR INHIBITING ADAM8 BIOLOGICAL ACTIVITIES”, Atty Dkt 3217/6 PROV, which is hereby incorporated by reference in its entirety.

### REFERENCE TO A “SEQUENCE LISTING,” APPENDIX SUBMITTED AS AN ASCII TEXT FILE

This application contains a sequence listing appendix. It has been submitted electronically via EFS-Web as an ASCII text file entitled “722-02-PCT_2020-05-11_SEQ_ST25.txt”. The sequence listing is 60,730 bytes in size, and was created on May 11, 2020. It is hereby incorporated by reference in its entirety.

### 1. Field

The presently disclosed subject matter relates to compositions and methods pertaining to the inhibition of ADAM8. In particular, the presently disclosed subject matter relates to modified and purified prodomain of ADAM8, to mutations of prodomain polypeptides, and to modifications that stabilize prodomains, and nucleic acids encoding ADAM8 modulating peptides, for in vitro and vivo use. The presently disclosed subject matter further relates to the use of prodomains in cellular assays, and to the use of prodomains, including gene therapy or cell-based therapies expressing prodomains, for treatment of diseases such as cancer, vascular diseases, fibrosis, wounds, asthma, allergies, lung injuries, and diseases related to excessive osteoclast formation or activity.

### 2. Background

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly nor impliedly admitted as prior art against the present disclosure.

ADAM8 is a member of the a disintegrin and metalloproteinase (ADAM) family (Edwards et al., 2008) that includes enzymes such as TACE (ADAM17), ADAM9, ADAM8, and ADAM10. In total, for humans, there are 33 ADAM family members. The ADAM proteins comprise a prodomain that is important for proper folding and transport of the enzyme through the cell, a catalytic domain containing a typical HEXXH motif, a disintegrin domain, that is used to interact with integrins, a cysteine rich region that is believed to be important for substrate recognition, a transmembrane domain, and a cytoplasmic tail that is involved in signaling events. See FIG. 1-FIG. 3.

Members of the ADAM family are known to cleave type I and type II single membrane spanning proteins from cells to generate soluble mature proteins that have varying physiological roles (Edwards et al., 2008). For example, TACE is known to generate soluble epidermal growth factor (EGF) ligands such as TGF-alpha, amphiregulin, and HB-EGF (Sahin et al., 2004). Similarly, ADAM10 activity generates soluble proteins including, but not limited to, EGF ligands, EGF, HB-EGF, and betacellulin (Sahin et al., 2004), Notch, amyloid precursor protein, ephrins, cadherins, protocadherins, chemokines such as CXCL16 and CX3CL1, HER2, AXL, cMET, and CD23, a low affinity receptor for IgE (reviewed in Pruessmeyer & Ludwig, 2009). Excess ADAM9 activity may promote cell invasiveness and growth in tumor cell assays due to enhanced production of soluble epidermal growth factor (EGF) ligands and MT-MMP16 (Moss et al., 2011). In addition, ADAM9 activity is linked to neo-vascularization events through processing of Tie-2 and other factors and VEGFR2 (Guaiquil et al., 2009; Maretzky et al., 2017), and plays a protective role in wound healing (Mauch et al, 2010), acute lung injury (Roychaudhuri et al., 2014), and COPD (Wang et al., 2018) as ADAM9 knockout mice fare better than their wild type counterparts. In addition, inhibition of ADAM9 increases ADAM10 activity which promotes formation of soluble amyloid precursor protein-alpha which is linked to helping people with Alzheimer's disease (Moss et al., 2011). Inhibition of ADAM9 is also linked to increases in BMP7, and decreases in TGF-beta RI and activin RI which is linked to fibrosis, and also decreases in MAC-1 and lymphotactin.

ADAM8 undergoes autocatalysis to be activated. Cleavage of the prodomain to release active ADAM8 occurs at a furin recognition motif (41-44) by furin and at amino acids near or at glutamate 158 and glutamate 161 by ADAM8 itself (Hall et al., 2009). See FIG. 4A and FIG. 4B. ADAM8 does not have a furin site at the junction between the prodomain and catalytic domain, which is unlike other ADAM family members. Glycosylation of the prodomain affects the autocatalytic activation of ADAM8 as mutation of one of its glycosylation sites, N91, impairs trafficking of active ADAM8 to the cell surface as well as processing of the prodomain (Srinivasin et al., 2014). In the trans golgi network, the prodomain while it is still attached to ADAM8 is degraded. Therefore, there is never any intact prodomain produced. Once the prodomain is cleaved at these sites, the truncated prodomains are degraded.

ADAM8 exists as both membrane and soluble forms and both forms have been implicated in disease states such as triple negative breast cancer, pancreatic cancer, asthma, vascular disease, asthma and allergic responses, lung injury, diseases of the bone due to promoting osteoclast formation such as osteoporosis, periprosthetic osteolysis, bone tumors, Paget's disease, and impairing re-vascularization. ADAM8 also cleaves CD23 as well as L-selectin and other substrates such as TNFR2 (Table 1). These findings suggest that treating individuals with an ADAM8 inhibitor would be beneficial to certain cell proliferative, invasive, and pro-inflammatory conditions.

Accordingly, the ability to specifically modulate ADAM8 activity would be useful to study the biological functions of the protein, and for the treatment of disorders including but not limited to cancer, asthma, allergies, lung injuries, diseases related to excessive osteoclast formation or activity, vascular diseases, wound healing and fibrosis

Unfortunately, existing small molecule inhibitors are not specific for ADAM8 activity or lack acceptable pharmacokinetic properties. For example, hydroxamates developed by GSK inhibit many ADAM members as well as other members of the matrix metalloproteinase family (Ludwig et al., 2005). Inhibitors disclosed by Incyte also inhibit MMPs, and possibly other ADAM family members (Zhou et al., 2006). Such non-specific inhibition often leads to unwanted side effects, and in this case has prevented the compounds from being developed into pharmaceutical drugs (Moss et al., 2008). A small molecule that binds to the disintegrin domain of ADAM8 works well in cancer and other models but has a short half-life in vivo.

ADAM family members are expressed as zymogens with the prodomains maintaining the enzymes in a latent state. For example, the prodomain of TACE suppresses the activity of its catalytic domain with a Kᵢ of 50 nM and inhibits TACE activity in vivo (Wong et al., 2016). The wild type prodomain of TACE, however, does not have good pharmacokinetic properties. Mutant prodomains that are modified an upstream furin site and cysteine residue stabilized the TACE prodomain for in vivo use (Wong et al., 2016). Likewise, the wild type prodomain of ADAM10 does not have good pharmacokinetic properties, thereby making it difficult to be used as a drug. The poor pharmacokinetics may be due to processing by a furin convertase at the upstream and down-stream cleavage sites. Furthermore, modifications to stabilize these sites improves IC50 values in cell-based assays. In contrast, modification of the only furin site in the prodomain of ADAM9, only improves IC50 values in cell based assays marginally. In addition, the sole cysteine at position 173 could interfere with the ability of the prodomain to have good pharmacokinetic properties as it can undergo oxidation to form a dimeric form of the prodomain. Both ADAM10 and ADAM9 prodomains have several meprin beta sites. ADAM10 has been shown to be a substrate for Meprin beta (Bergin et al., 2008; Deuss et al. 2008; Banerjee et al., 2011; Vazeille et al., 2011; Schutte et al., 2014), and inhibition of meprin beta could have unwanted side effects (Jefferson et al., 2013).

For ADAM8, it has an upstream and downstream furin site, three cysteines, and a downstream site where it is auto-catalytically cleaved. These features of the prodomain make it unstable for in vitro and in vivo use.

Accordingly, there is a need in the art for selective inhibitors of ADAM8 to study the biological functions of the proteins and to treat diseases and disorders such as but not limited to cancer, asthma, allergies, lung injuries, diseases related to excessive osteoclast formation or activity, vascular disease, wounds, and fibrosis.

### 3. SUMMARY OF THE DISCLOSURE

In some embodiments, the presently disclosed subject matter relates to peptides comprising, consisting essentially of, or consisting of the amino acid sequence set forth in SEQ ID NO: 3, wherein relative to the amino acid sequence set forth in SEQ ID NO: 2, the peptide includes one or more amino acid substitutions and/or modifications (e.g., chemical modifications of the sulfhydryl groups of one or more of the cysteines present in SEQ ID NO: 2) at an amino acid position selected from the group consisting of amino acids 35, 41, 43, 44, 105, 124, 152-164, and 167 of SEQ ID NOS: 1, 12, OR 13 (corresponding to amino acids 20, 26, 28, 29, 90, 109, 137-149, and 152 of SEQ ID NO: 2 or SEQ ID NO: 3) such that the peptide is not auto-catalytically cleaved, is less sensitive to furin cleavage, is less susceptible to oxidation and/or disulfide bond formation, or any combination thereof, as compared to a peptide without the one or more amino acid substitutions and/or modifications. In some embodiments, the peptide comprises, consists essentially of, or consists of an amino acid sequence that is at least 90% identical to SEQ ID NO: 2. In some embodiments, as compared to SEQ ID NOS: 1, 12, OR 13, the amino acid sequence of the peptide comprises one or more amino acid substitutions and/or modifications selected from the group consisting of a substitution of tryptophan 35 to another amino acid, optionally arginine; a substitution of arginine 41 to another amino acid, optionally alanine, serine, glycine, or lysine; a substitution of arginine 43 to another amino acid, optionally alanine, serine, glycine, or lysine; a substitution of arginine 44 to another amino acid, optionally alanine, serine, glycine, or lysine; a substitution of cysteine 105 to another amino acid, optionally serine, alanine, or glycine, a substitution of cysteine 124 to another amino acid, optionally serine, alanine, or glycine, a substitution of cysteine 167 to another amino acid, optionally serine, alanine, or glycine, and a chemical modification of one, two, or all three of cysteines 105, 124, and 167, or any combination thereof. In some embodiments, the peptides of the presently disclosed subject matter consists or comprises modification of glutamine 156, glutamate 158, or glutamine 162. In other embodiments any combination of the three residues (glutamine 156, glutamate 158, glutamine 162) are modified with any amino acid to render the peptide more resistant to cleavage by auto-catalysis. In some embodiments any combination of amino acids ranging from 152-164 are modified with any amino acids to render the peptide more resistant to cleavage by auto-catalysis.

In some embodiments, the peptides of the presently disclosed subject matter further comprise a pegylated cysteine added to the N-terminus, to the C-terminus, or both, optionally wherein one or both of the pegylated cysteines comprise a PEG group having a molecular weight of about 1 kiloDalton (kDa) to about 40 kDa. In some embodiments, as compared to SEQ ID NOS: 1, 12, OR 13, the amino acid sequence comprises, consists essentially of, or consists of a substitution of at least one of arginine 41, arginine 43, and arginine 44 to an amino acid other than arginine, and substitutions of cysteines 105, 124, and 167 to serine. In some embodiments, as compared to SEQ ID NOS: 1, 12, OR 13, the amino acid at positions 105, 124, and 167 are derivatized with something to improve solubility, pharmacokinetics, stability, potency, reduce immunogenicity or to improve refolding from inclusion bodies, optionally wherein one or more cysteines at positions 105, 124, or 167 comprises a PEG group having a molecular weight of about 1 kDa to about 40 kDa. In some embodiments, as compared to SEQ ID NOS: 1, 12, OR 13, one or more of cysteines 105, 124, and 167 comprises a chemical modification with a maleimide ester or acyl halide.

In some embodiments, the peptides of the presently disclosed subject matter further comprise modifications of arginine 41 of SEQ ID NOS: 1, 12, OR 13 or a corresponding position in any of SEQ ID NOs: 2-9, arginine 43 or a corresponding position in any of SEQ ID NOs: 2-9, and/or arginine 44 or a corresponding position in any of SEQ ID NOs: 2-9 or a plurality of modifications at these positions to increase resistance of the peptide to furin cleavage. In some embodiments, the modifications of arginine 41 or a corresponding position in any of SEQ ID NOs: 2-9, arginine 43 or a corresponding position in any of SEQ ID NOs: 2-9, and/or arginine 44 or a corresponding position in any of SEQ ID NOs: 2-9 are independently selected at each amino acid from the group consisting of substitutions with any amino acid other than cysteine and chemical modifications, in some embodiments chemical modifications that modify the sulfhydryl group of the cysteine to a group that is less resistant to oxidation. In some embodiments, one or more of cysteines 105, 124, and 167 or corresponding positions in any of SEQ ID NOs: 2-9 comprises a chemical modification resulting from reacting the one or more cysteines with a disulfide. In some embodiments, tryptophan 35 or a corresponding position in any of SEQ ID NOs: 2-9 is replaced with arginine. In another embodiment, tryptophan 35 or a corresponding position in any of SEQ ID NOs: 2-9 is replaced with arginine, the cysteines, amino acids 105, 124, and 167 or corresponding positions in any of SEQ ID NOs: 2-9, are replaced with serines, and amino acid 44 or a corresponding position in any of SEQ ID NOs: 2-9 is replaced with alanine or glycine.

In some embodiments, the peptides of the presently disclosed subject matter comprise, consist essentially of, or consist of an amino acid sequence having a percent identity of at least 87% to any one of SEQ ID NOS: 3-9 or 14-20, optionally wherein the percent identity is at least 93%, and further optionally wherein the percent identity is at least 95%, 96%, 97%, 98%, 99%. In some embodiments, the peptide comprises, consists essentially of, or consists of an amino acid sequence having 100% percent identity to any one of SEQ ID NOs: 3-9 or 14-20 over its full length.

In some embodiments, the peptides of the presently disclosed subject matter further comprise one or more additional modifications selected from the group consisting of conservative amino acid substitutions, non-natural amino acid substitutions, D- or D,L-racemic mixture isomer form amino acid substitutions, amino acid chemical substitutions, carboxy- and/or amino-terminus modifications, glycosylations, and conjugations to biocompatible molecules such as but not limited to fatty acids and other peptides of interest.

In some embodiments, the peptides of the presently disclosed subject matter the peptide further comprise a modification of at least one of cysteines 105, 124, and/or 167 or corresponding positions in any of SEQ ID NOs: 2-9 or 14-20, wherein the modification comprises attachment of a maleimide ester derivative comprising at least one moiety selected from the group consisting of a PEG group, a fluorescent moiety, an alkyl moiety, a colorimetric moiety, a bifunctional moiety, a radiometric moiety, a glycosyl moiety, a carbohydrate, a fatty acid moiety, a toxin, a therapeutic agent, optionally a chemotherapeutic agent, a linker, a peptide of interest, or any combination thereof.

In some embodiments, the presently disclosed subject matter also relates to compositions comprising the peptides disclosed herein. In some embodiments, the compositions are formulated for administration to a subject or are pharmaceutical compositions formulated for administration to a human.

In some embodiments, the presently disclosed subject matter also relates to fusion proteins that comprise a peptide as disclosed herein. In some embodiments, the fusion protein comprises the peptide conjugated to an agent selected from the group consisting of a therapeutic moiety, a diagnostic moiety, a detectable moiety, or any combination thereof, optionally wherein the peptide is conjugated to the agent via a linker molecule or via a peptide linkage. In some embodiments, the therapeutic molecule is selected from the group consisting of a therapeutic antibody, an Fc fragment, a nanobody, a receptor, a toxin, a chemotherapeutic molecule, or any combination thereof.

In some embodiments, the presently disclosed subject matter also relates to polypeptides comprising, consisting essentially of, or consisting of an amino acid sequence as set forth in any of SEQ ID NOs 3-9 or 14-20. In some embodiments, the polypeptide does not comprise SEQ ID NO: 2, meaning that as compared to SEQ ID NO: 2, the amino acid sequence comprises at least one substitution, chemical modification, or any combination thereof. In some embodiments, the at least one substitution, chemical modification, or any combination thereof is at a position other than methionine 1 of any of SEQ ID NOs: 2-9 or 14-20.

In some embodiments, the polypeptides of the presently disclosed subject matter further comprise a tag, optionally a His tag, that can be employed for purification and/or isolation of the polypeptide from an expression system.

In some embodiments, the polypeptides of the presently disclosed subject matter further comprise a recognition site for a protease between the tag and an amino acid of the polypeptide that can be employed for releasing the tag from the polypeptide by proteolytic cleavage.

In some embodiments, the presently disclosed subject matter also relates to polypeptides comprising an amino acid sequence as set forth in any one of SEQ ID NO: 2-9 or 14-20, and further comprise an addition of one or more amino acids to the N-terminus of the polypeptide, an addition of one or more amino acids to the C-terminus of the polypeptide, or an addition of one or more amino acids to both the N-terminus and the C-terminus of the polypeptide, wherein the one or more amino acids comprises at least one cysteine residue that provides functionality to conjugate a moiety of interest to the polypeptide. In some embodiments, the one or more amino acids added to the N-terminus of the polypeptide comprise, consist essentially of, or consists of SEQ ID NO: 10 or SEQ ID NO: 11 and or the one or more amino acids added to the C-terminus of the polypeptide comprise, consist essentially of, or consists of SEQ ID NO: 10 or SEQ ID NO: 11. In some embodiments, the polypeptide further comprises a PEG group conjugated to a cysteine present in the one or more amino acids added to the N-terminus and/or the C-terminus of the polypeptide, wherein the PEG group enhances the proper folding of the polypeptide and/or stabilizes the polypeptide to auto-catalytic or furin cleavage relative to the polypeptide lacking a PEG group.

In some embodiments, the presently disclosed subject matter also relates to methods for modulating ADAM8 biological activities in vitro. In some embodiments, the methods comprise contacting a solution or a cell comprising an ADAM8 protein with a peptide or a composition as disclosed herein in an amount sufficient to inhibit the activity of the ADAM8 protein.

In some embodiments, the presently disclosed subject matter also relates to methods for inhibiting an ADAM8 biological activity in a subject. In some embodiments, the methods comprise administering to a subject a peptide or a composition as disclosed herein in an amount and via a route sufficient to contact an ADAM8 polypeptide present in the subject, whereby an ADAM8 biological activity in a subject is modulated.

In some embodiments, the presently disclosed subject matter also relates to methods for inhibiting ADAM8 biological activities in vivo. In some embodiments, the methods comprise administering to a subject a polypeptide comprising, consisting essentially of, or consisting of an amino acid sequence as set forth in any one of SEQ ID NO: 2-9 or 14-20 in an amount and via a route sufficient to inhibit an ADAM8 biological activity in vivo, optionally wherein the polypeptide is pegylated.

In some embodiments, the presently disclosed subject matter also relates to methods for inhibiting ADAM8 biological activities associated with diseases or disorders in subjects. In some embodiments, the methods comprise contacting an ADAM8 protein present in a subject with an effective amount of a peptide or a composition as disclosed herein, wherein the disease or disorder is selected from the group consisting of cancer, inflammation, vascular disease, fibrosis, asthma, allergies, a wound, and undesirable angiogenesis, or wherein the subject has a predisposition thereto. In some embodiments, the disease or disorder comprises lung injury, optionally lung injury and the effective amount is sufficient to reduce elastin degradation, inflammation, a matrix metalloprotein biological activity, or any combination thereof in one or both of the subject's lungs. In some embodiments, the disease or disorder comprises a liver injury, optionally a liver injury associated with liver fibrosis, and the effective amount is sufficient to reduce a biological activity of an MMP gene product, in the subject's liver. In some embodiments, the disease or disorder results at least in part from excess cell proliferation associated with an ADAM8 biological activity. In some embodiments, the subject has a disease or disorder characterized at least in part by undesirably high ADAM8 biological activity or ADAM8 protein expression.

In some embodiments, the presently disclosed subject matter also relates to methods for decreasing inflammation. In some embodiments, the methods comprise administering to a subject an effective amount of a peptide or a composition as disclosed herein.

In some embodiments, the presently disclosed subject matter also relates to methods for inhibiting cell invasion associated with undesirable ADAM8 biological activities in subjects. In some embodiments, the methods comprise administering to a subject in need thereof an effective amount of a peptide or a composition as disclosed herein.

In some embodiments, the presently disclosed subject matter also relates to methods for inhibiting the release of a substrate of ADAM8 in vivo or degradation of a substrate of ADAM8 in vivo. In some embodiments, the method comprises administering to a subject in need thereof a peptide comprising, consisting essentially of, or comprising an amino acid sequence as set forth in any one of SEQ ID NO: 2-9 or 14-20 optionally wherein the peptide is pegylated, or where the peptide is modified to improve potency, solubility, pharmacokinetics, pharmacodynamics, reduce immunogencity, improve refolding or any combination thereof.

In some embodiments of the presently disclosed methods, the peptide or the composition is formulated for administration via a route selected from the group consisting of inhalation, oral administration, intraadiposal administration, intraarterial administration, intraarticular administration, intracranial administration, intradermal administration, intralesional administration, intramuscular administration, intranasal administration, intraocular administration, intrapericardial administration, intraperitoneal administration, intrapleural administration, intraprostatic administration, intrarectal administration, intrathecal administration, intratracheal administration, intratumoral administration, intraumbilical administration, intravaginal administration, intravenous administration, intravesicular administration, intravitreal administration, subconjunctival administration, subcutaneous administration, sublingual administration, topical administration, transbuccal administration, oropharyngeal aspiration, and transdermal administration. In some embodiments, the peptide or the composition is delivered in a lipid composition, optionally a liposome or an ethosome; in a creme; via a catheter; via lavage; via infusion, optionally via continuous infusion; via inhalation; via injection; via local delivery; via localized perfusion; by bathing target cells directly; or any combination thereof.

In some embodiments, the presently disclosed subject matter also relates to methods for attaching a peptide consisting of, consisting essentially of, or comprising an amino acid sequence as set forth in any of SEQ ID NOs 2-9 or 14-20 to an antibody, antibody fragment, or other protein, the method comprising conjugating the peptide to the antibody, antibody fragment, or other protein, optionally via a linker, further optionally via a peptide linker

In some embodiments, the presently disclosed subject matter also relates to peptides comprising, consisting essentially of, or consisting of any of SEQ ID NOs 2-9 or 14-20, wherein at least one ADAM8 autocatalytic cleavage site present therein is modified to render the peptide less susceptible to autocatalyis wherein the at least one cleavage site is selected from the group consisting of amino acids 137-149 of SEQ ID NOS: 3, 19, or 20, and further optionally wherein each X is independently selected from the group consisting of any of the 20 naturally occurring amino acids.

In some embodiments, the presently disclosed subject matter also relates to polypeptides comprising, consisting essentially of, or consisting of an amino acid sequence as set forth in any one of SEQ ID NOS: 2-9 or 14-20, wherein one or more cysteines that correspond to amino acid residues of SEQ ID NO: 2 selected from the group consisting of cysteines 105, 124, and 167 of SEQ ID NOS: 1, 12, OR 13 or corresponding positions of any of SEQ ID NOs: 2-9 or 14-20, or any combination thereof, is/are conjugated to one or more moieties that improve the inhibitory potency, solubility, and/or a pharmacokinetic property of the polypeptide relative to the polypeptide lacking the moiety, and/or is conjugated to one or more chromophores, fluorophores, and/or radionucleotides.

In some embodiments, the presently disclosed subject matter also relates to polypeptides comprising, consisting essentially of, or consisting of an amino acid sequence as set forth in one of SEQ ID NOs 2-9 or 14-20, for use in treating a subject with a disorder associated with undesirable ADAM8 biological activity, optionally wherein the disorder associated with undesirable ADAM8 biological activity is selected from the group consisting of cancer, inflammation, fibrosis, asthma, allergies, lung injuries, diseases related to excessive osteoclast formation or activity, and undesirable angiogenesis, optionally wherein the polypeptide is pegylated at one or more of the amino acids of SEQ ID NOs 2-9 or 14-20 or after a cysteine has been introduced to the sequence. In some embodiments, the polypeptide is pegylated. In some embodiments, the subject is a human.

In some embodiments, the presently disclosed subject matter also relates to uses of inhibitors of ADAM8 biological activities to treat subjects with disorders associated with undesirable ADAM8 biological activities. In some embodiments, the inhibitor of the ADAM8 biological activity is selected from the group consisting of an antibody or a fragment thereof, optionally wherein the antibody is a monoclonal antibody, a protein, a peptide, an inhibitory nucleic acid, and/or a small molecule, optionally wherein the disorder associated with undesirable ADAM8 biological activity is selected from the group consisting of cancer, inflammation, asthma, allergies, lung disorders, lung injuries, diseases related to excessive osteoclast formation or activity, fibrosis, a wound, and undesirable angiogenesis. In some embodiments, the subject is a human

In some embodiments, the presently disclosed subject matter also relates to polypeptides comprising, consisting essentially of, or consisting of an amino acid sequence as set forth in one of SEQ ID NOs 2-9 or 14-20, for use in preventing development of and/or reducing the severity of at least one symptom of a disorder associated with an undesirable ADAM8 biological activity. In some embodiments, the disorder associated with undesirable ADAM8 biological activity is selected from the group consisting of cancer, inflammation, fibrosis, asthma, allergies, lung injuries, diseases related to excessive osteoclast formation or activity, a wound, and undesirable angiogenesis, in a subject in need thereof, optionally wherein the subject has a predisposition to development the at least one symptom. In some embodiments, the polypeptide is pegylated. In some embodiments, the subject is a human. In some embodiments, the disorder associated with undesirable ADAM8 biological activity is selected from the group consisting of fibrosis, optionally wherein the fibrosis is liver fibrosis.

In some embodiments, the presently disclosed subject matter also relates to peptides comprising, consisting essentially of, or consisting of any of SEQ ID NOs 2-9 or 14-20, wherein at least one furin or furin-like cleavage site present therein is modified to render the at least one furin or furin-like cleavage site more resistant to cleavage by a furin or furin-like convertase as compared to the at least one furin or furin-like cleavage site that is unmodified. In some embodiments, the at least one furin or furin-like cleavage site consists of the tetrapeptide sequence RXRR, RXKR, RXXR, or RXRK, where X is any amino acid.

In some embodiments, the presently disclosed subject matter also relates to polypeptides comprising, consisting essentially of, or consisting of an amino acid sequence as set forth in any one of SEQ ID NOs 2-9 or 14-20, wherein the amino acid sequence comprises a substitution of one or more charged amino acids relative to SEQ ID NOS: 1, 12, OR 13 such that the solubility of the polypeptide is increased in a pre-determined solvent relative to a polypeptide comprising, consisting essentially of, or consisting of an amino acid sequence as set forth in SEQ ID NOS: 1, 12, OR 13, and further wherein the pre-determined solvent is selected from the group consisting of a biological fluid and a cell culture medium.

In some embodiments, the presently disclosed subject matter also relates to polypeptides comprising an amino acid sequence as set forth in any one of SEQ ID NOs 2-9 or 14-20 and further comprising a C-terminal spacer containing one or more charged residues, optionally selected from the group consisting of Asp, Glu, Arg, and Lys, wherein solubility of the polypeptide with the C-terminal spacer with respect to a pre-determined solvent is greater than that of the same polypeptide absent the C-terminal spacer.

In some embodiments, the presently disclosed subject matter also relates to polypeptides comprising an amino acid sequence as set forth in any one of SEQ ID NOs 2-9 or 14-20 and further comprising an N-terminal spacer containing one or more charged residues, optionally selected from the group consisting of Asp, Glu, Arg, and Lys, wherein solubility of the polypeptide with the N-terminal spacer with respect to a pre-determined solvent is greater than that of the same polypeptide absent the N-terminal spacer.

In some embodiments, the presently disclosed peptides, polypeptides, and/or fusions proteins of the presently disclosed subject matter are modified by amino acid substitutions, chemical modifications, or combinations thereof such that the peptides, polypeptides, and/or fusions proteins are less susceptible to cleavage by autocatalyis as compared to the peptides, polypeptides, and/or fusions proteins that lack the modification(s). In some embodiments, the modifications are modifications of one or more of cysteines 105, 124, and/or 167 of SEQ ID NOS: 1, 12, OR 13 or corresponding positions of any of SEQ ID NOs: 2-9 or 14-20 or at a position in the peptides, polypeptides, and/or fusions proteins that corresponds to one or more of cysteines 105, 124, and/or 167 of SEQ ID NOS: 1, 12, OR 13 or corresponding positions of any of SEQ ID NOs: 2-9 or 14-20.

In some embodiments, the presently disclosed peptides, polypeptides, and/or fusions proteins of the presently disclosed subject matter are present in a composition, optionally a pharmaceutical composition, wherein the composition is formulated for administration to a subject or is a pharmaceutical composition formulated for administration to a human

In some embodiments, the presently disclosed peptide of SEQ ID NO: 3, 19, or 20 have one, two, three, four, or more additional amino acid mutations at autocatalysis sites.

In some embodiments the presently disclosed peptides of SEQ ID NO: 2-9 or 14-20 (derivatized or not) are used in combination to treat diseases characterized by excessive ADAM8 activity or diseases where inhibition of ADAM8 can be therapeutic such as cancer, inflammation, asthma, allergies, lung injuries, diseases related to excessive osteoclast formation or activity, Alzheimers, vascular diseases, fibrosis, treatment of wounds.

An object of the presently disclosed subject matter having been stated above, other objects and advantages will become apparent upon a review of the following Detailed Description and Examples.

### 4. BRIEF DESCRIPTION OF THE SEQUENCE LISTING

SEQ ID NO: 1 is the amino acid sequence of full length human ADAM8 as set forth in Accession No. NP_001100.3 of the GENBANK® biosequence database.

SEQ ID NO: 2 is the amino acid sequence of an exemplary human ADAM8 prodomain peptide, which comprises amino acids 17-196 of SEQ ID NOS: 1, 12, OR 13 with and N-terminal methionine.

SEQ ID NO: 3 is a consensus core amino acid sequence of the modified ADAM8 prodomain peptides of the presently disclosed subject matter. It is based on SEQ ID NO: 2 and differs from SEQ ID NO: 2 by virtue of the positions at which the modified ADAM8 prodomain peptides of the presently disclosed subject matter differ from SEQ ID NO: 2 within its core denoted by “X”, wherein X is any amino acid or a modification thereof.

SEQ ID NO: 4 is SEQ ID NO: 2 with amino acid 20, the cysteines, and the furin site modified.

SEQ ID NOs: 5 and 6 are SEQ ID NO: 2 with the cysteines and the furin site modified.

SEQ ID NOs: 7 and 8 are SEQ ID NO: 2 with the cysteines, the furin site, and the auto-catalytic site modified.

SEQ ID NO: 8 is SEQ ID NO: 2 with the cysteines, the furin site, and the auto-catalytic site modified.

SEQ ID NO: 9 is SEQ ID NO: 2 with amino acid 20, the cysteines, the furin site, and the auto-catalytic site modified.

SEQ ID NOs: 10 and 11 are exemplary pentapeptide sequences that can be added to the N-terminus and/or the C-terminus of a modified ADAM8 prodomain peptide or polypeptide of the presently disclosed subject matter as a monomer, dimer, trimer, or multimer. It is noted that the precise amino acid sequence of the added amino acids can differ from SEQ ID NOs: 10 and 11 in one or more positions of one or more of the monomers, dimers, trimers, or multimers, if desired.

SEQ ID NO: 12 is a naturally occurring variant of SEQ ID NO:1 where tryptophan 35 is arginine.

SEQ ID NO:13 is a SEQ ID NO:1 where amino acid 35 is replaced with an X and X may be tryptophan or arginine.

SEQ ID NO:14 is SEQ ID NO:2 with amino acid 20 replaced with arginine.

SEQ ID NO:15 is SEQ ID NO:14 with the furin site and cysteines modified.

SEQ ID NO:16 is SEQ ID NO:14 with phenylalanine replaced with leucine at position 91.

SEQ ID NO:17 is SEQ ID NO:2 with the furin site and cysteines modified

SEQ ID NO:18 is another consensus core amino acid sequence of the modified ADAM8 prodomain peptides of the presently disclosed subject matter. It is based on SEQ ID NO: 2 and differs from SEQ ID NO: 2 by virtue of the positions at which the modified ADAM8 prodomain peptides of the presently disclosed subject matter differ from SEQ ID NO: 2 within its core denoted by “X”, wherein X is any amino acid or a modification thereof.

SEQ ID NO:19 is another consensus core amino acid sequence of the modified ADAM8 prodomain peptides of the presently disclosed subject matter where in relative to SEQ ID NO:18, the downstream ADAM8 cleavage sites are also denoted by “X” wherein X is any amino acid, or modification thereof.

SEQ ID NO:20 is another consensus core amino acid sequence of the modified ADAM8 prodomain peptides of the presently disclosed subject matter where relative to SEQ ID NO:19, the glycosylation sites are also denoted by “X” wherein X is any amino acid, or modification thereof.

SEQ ID NO:21-26 are optimized bacterial DNA sequences to express the polypeptides disclosed herein.

SEQ ID NO:27 is the DNA sequence for the naturally occurring human ADAM8 prodomain This DNA encodes the prodomain of the ADAM8 protein in SEQ ID NO:1.

### 5. BRIEF DESCRIPTION OF THE FIGURES

FIG. 1: The metzincin family of metalloproteinases. The MMP, ADAM, and ADAM-TS proteinases are members of the metzincin family of metalloproteinases. There are similarities to each other, especially in the catalytic domains where homologies between sub families can be as high as 30%. All family members contain prodomains that keep the enzymes in a latent state. Each member contains a consensus zinc binding domain (HEXXH), and a methionine turn (M). For substrate recognition, the MMPs contain hemopexin domains, the ADAMs contain disintegrin domains, and the ADAM-TS proteins contain thrombospondin motifs (TS). Small molecule inhibitors that have been made in the past, are not selective, prompting researchers to focus on protein therapeutics.

FIG. 2: Features of individual prodomains for some ADAM family members. ADAM family members are membrane bound metalloproteases, but can also be released to produce soluble forms. Depicted in FIG. 2 are the partial structures for ADAM17, ADAM10 and ADAM9 and how they differ from one another in their prodomains. ADAM family members exist as zymogens, where the prodomains keep the enzymes in an inactive state and are required for proper folding and chaperonin functions. For ADAM17, ADAM10, and ADAM9, furin like proteases release the prodomain from the rest of the enzymes during the maturation process. A further upstream furin site in the ADAM17 and ADAM9 prodomains (ADAM10 has two), are also cleaved by furin like proteinases. Mutation of both furin sites to stabilize them towards furin degradation in the ADAM10 prodomain improves the pharmacokinetic properties of the prodomain and additionally, dramatically improves its potency in cell based assays. For ADAM9, stabilizing the furin site slightly improves its potency in cell based assays.

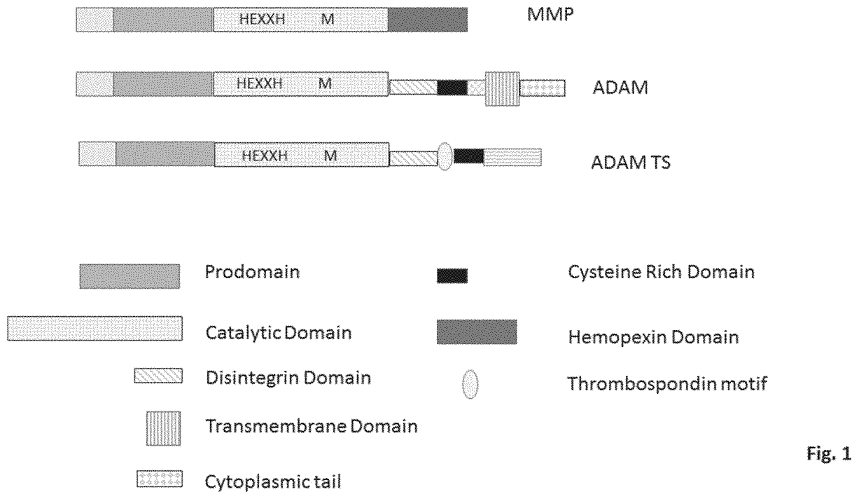

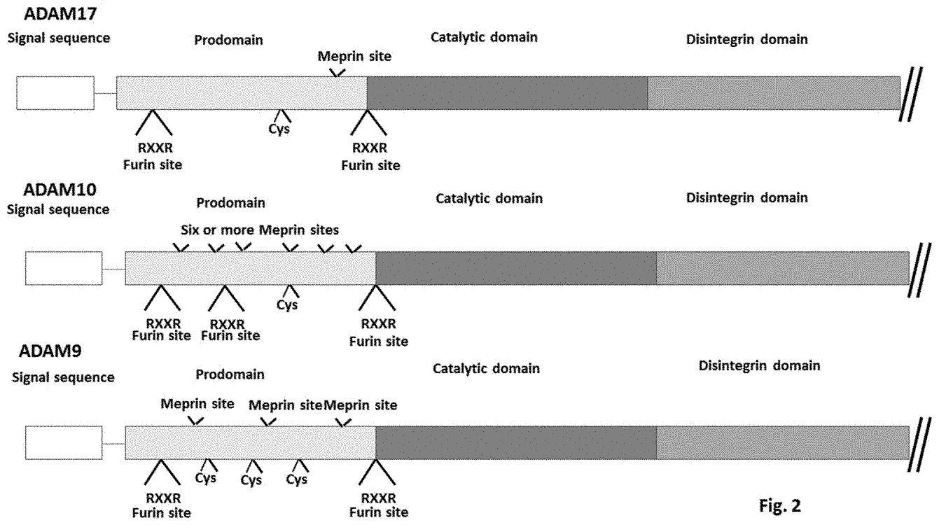

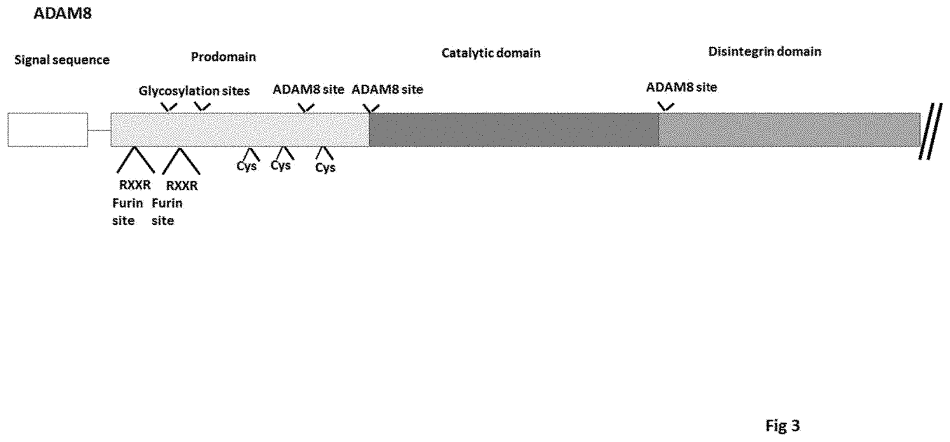

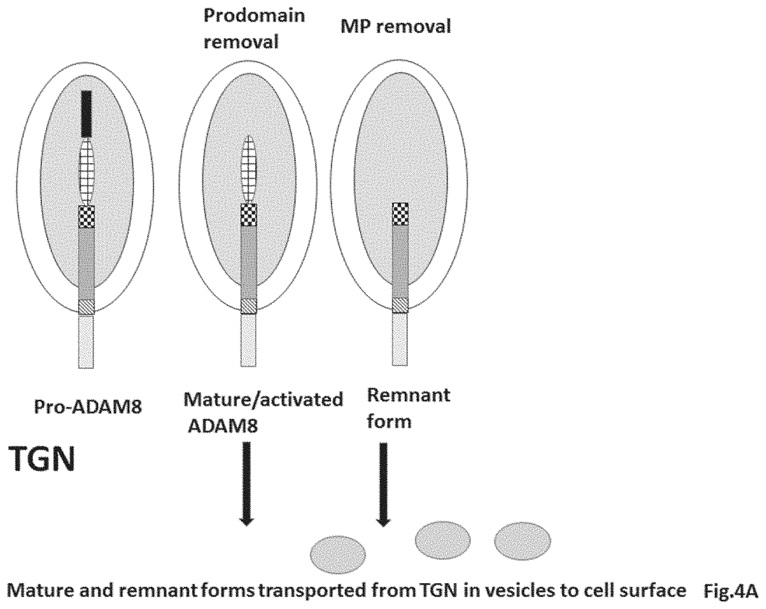

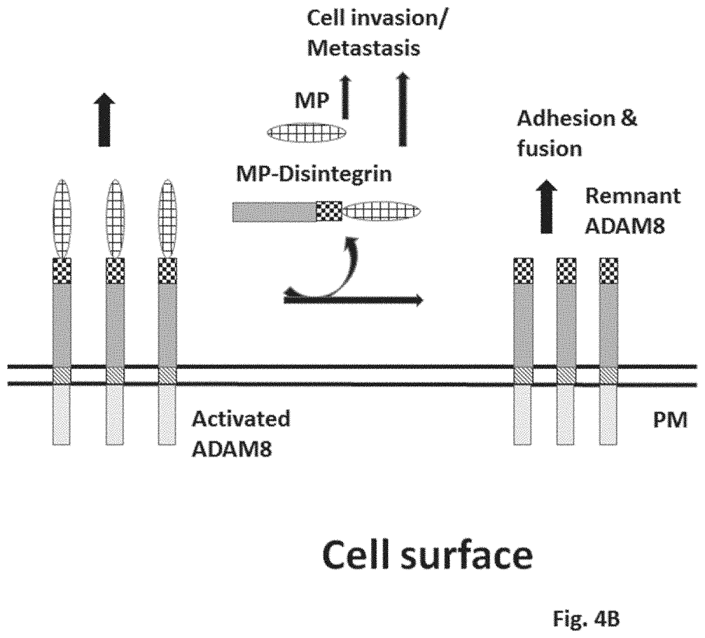

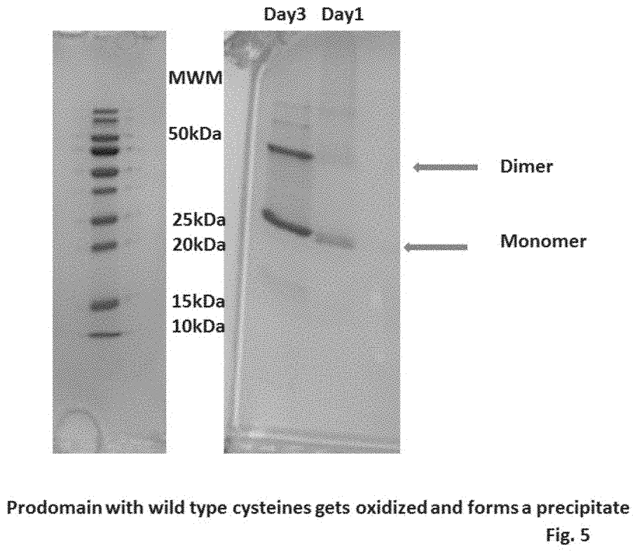

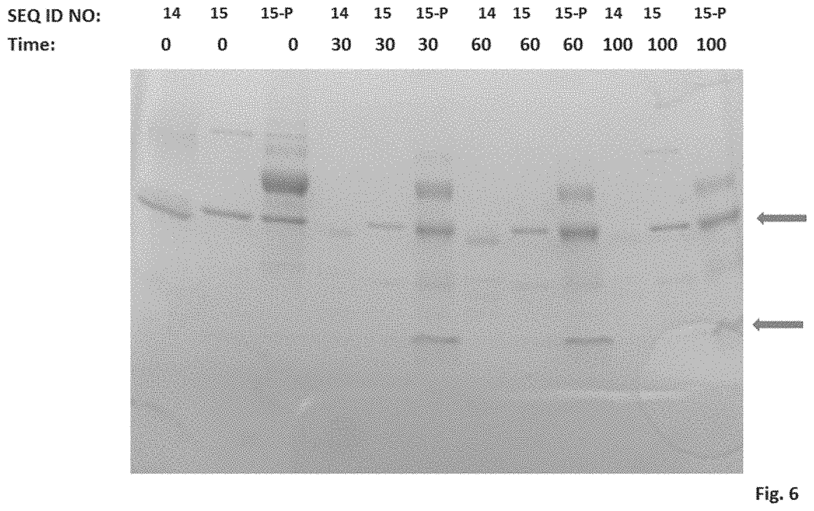

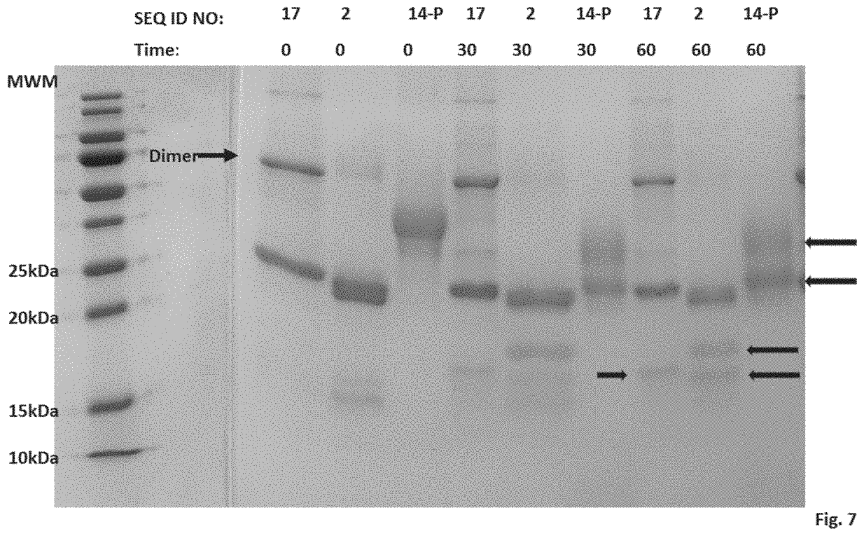

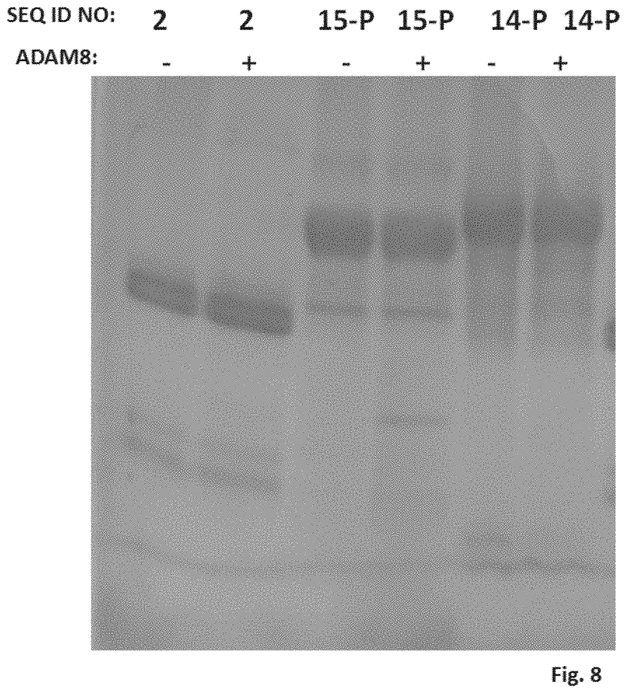

Both ADAM17 and ADAM10 contain 1 cysteine but ADAM9 contains 3 cysteines. Mutation of the cysteines to serine or alanine prevents oxidation and multimerization of the prodomains, while retaining or improving the inhibitory properties of the prodomains.

All of these ADAM metalloproteinases have meprin beta cleavage sites. Meprin beta can cleave all three prodomains in vitro, resulting in inactivation of the prodomains (they no longer inhibit their respective ADAM members). Meprin beta is much less efficient at cleaving the ADAM9 prodomains vs the ADAM10 prodomains. There are as many as 6 or more different meprin beta sites in the ADAM10 prodomain.

FIG. 3: The ADAM8 prodomain and its inactivation or degradation is unique. ADAM8 is very different in terms of its maturation process and the inhibitory properties of its prodomain. ADAM8 has no furin site between the prodomain and catalytic domain to release itself from its prodomain. Instead, ADAM8 autocatalytically cleaves itself by degradation of its prodomain, a step required for maturation before reaching the cell surface. ADAM8 also may auto-catalytically release its metalloproteinase domain and/or metalloproteinase and disintegrin domain, from the membrane to generate soluble forms. When the ADAM8 prodomain with its catalytic domain, or prodomain with the catalytic and disintegrin domain, is expressed in human cell lines, the isolated proteins begin with an amino acid N termini of 45, indicating that it is processed by a furin like proteinase before being released. Cleavage at the upstream furin site produces a prodomain that still retains inhibitory potency of the catalytic domain but not the catalytic and disintegrin domain We have also identified a novel downstream ADAM8 furin site. Mutations at the upstream site, reduce the efficiency of processing at the upstream site.

ADAM8 contains 3 cysteine residues. Mutation of the three cysteines makes the prodomain resistant to oxidation and precipitation and chemical modification, even with large polyethylene glycol (peg) groups attached. Mutation of the three cysteines also improves solubility and aggregation of the prodomains while retaining their inhibitory properties. Pegylation of the prodomains with the cysteine mutations at the C-terminus improves potency and solubility. Pegylation at the N -terminus destroys the potency of the prodomain towards ADAM8.

The prodomain is also glycosylated and mutation of the glycosylation site and N91 impairs prodomain removal and translocation to the cell surface. This disclosure includes polypeptides where either glycosylation site, N67 or N91 are mutated. Two of the sites in the prodomain where ADAM8 cleaves itself is at position Q158 and Q161.

FIG. 4A: Processing and maturation of ADAM8. In the trans golgi network (TGN), ADAM8 is produced as a zymogen, with its prodomain. The ADAM8 auto-catalytically degrades and releases its prodomain, and possibly its metalloproteinase domain (MP) leaving behind membrane bound activated ADAM8 and a remnant fragment that still contains the disintegrin where they are transported to the cell surface in vesicles.

FIG. 4B: The activated, membrane bound, ADAM8, is involved in processing of cell surface proteins called “shedding”. The ADAM8 can process itself further on the cell surface, to release the metalloproteinase domain (MP) or the metalloproteinase domain with its disintegrin domain intact (MP-Disintegrin). These soluble forms are involved in a variety of functions including cell migration and metastasis in cancer.

FIG. 5: ADAM8 prodomains are chemically unstable as they can undergo oxidation. A prodomain with the wild type cysteines, undergoes oxidation to form a dimer, during storage at 4° C. Also during storage, the prodomain precipitates. Similarly, ADAM8 prodomains that are modified to introduce cysteines undergo a dimerization and/or precipitation.

FIG. 6: A prodomain with wild type cysteines, SEQ ID NO:14, a mutant with a stabilized upstream furin site (R44G), containing a free cysteine at the C-terminus (SEQ ID NO: 15), or the pegylated version of SEQ ID NO: 15, was reacted with furin for varying lengths of time, and the reaction products were analyzed by SDS PAGE. The prodomain with no furin mutations (SEQ ID NO:14) was completely processed in 30min, whereas pegylated SEQ ID NO: 15 was resistant to furin cleavage and un-pegylated SEQ ID NO: 15 was completely resistant to furin degradation during the course of this experiment, suggesting that mutations at the upstream site, help prevent processing at the downstream site.

FIG. 7: Wild type prodomain (SEQ ID NO:2), a furin mutant (R41K and R44K), with three cysteines mutated (SEQ ID NO: 17), but with an introduced cysteine at the N-terminus, and a prodomain (SEQ ID NO: 14) with no furin mutations, but with all three cysteines pegylated, were reacted with furin for varying lengths of time, and the reaction products were analyzed by SDS PAGE. Both the wild type prodomain and pegylated SEQ ID NO: 14, were processed by furin at approximately the same rate at the upstream furin site (41-44) and a novel downstream site (73-77). The furin/cysteine mutant (SEQ ID NO:17), was processed more slowly, and only at the downstream site.

FIG. 8: Wild type prodomain (SEQ ID NO:2), pegylated SEQ ID NO:14, or pegylated SEQ ID NO:15 were reacted with and without ADAM8 (90 nM) for 1 hr at 37° C. and then subjected to SDS PAGE for analysis. These results show that the prodomains are stable to ADAM8 processing and/or degradation under the conditions used in the experiment.

### 6. DETAILED DESCRIPTION OF THE DISCLOSURE

The presently disclosed subject matter relates in some embodiments to modified ADAM8 modulating peptides and related compositions useful for studying the biological functions of ADAM8 and/or for the treatment of diseases and disorders associated with undesirable ADAM8 biological disease, a wound, a vascular disease, asthma, allergies, lung injuries, diseases related to excessive osteoclast formation or activity, and undesirable angiogenesis and disorders characterized at least in part by the presence of one or more of inflammation, excess cell proliferation, angiogenesis, fibrosis, and excess or decreased soluble proteins as described in Table 1.

TABLE 1
Exemplary Proteins Impacted by ADAM8 Modulating Peptides
Factors Increased by ADAM8 Overexpression
N-Cadherin / Jagged 1; ErbB2/HER2; E-Cadherin; Clusterin; Endoglycan; CHL-
1/L1CAM-2; ErbB3/HER3; CD40/TNFRS F5 ; VEGF R1; Integrin αV/CD51; NrCAM;
ErbB4/HER4; E-Selectin; VCAM-1; LRP-6; BMPR-IB/ALK-6; EpCAM/TROP-1; VEGF
R2/; EGF R/ErbB1; MCAM/CD146; Notch
Factors Decreased by ADAM8 Inhibition
CD23; L-selectin; MMP9; TNF RII/TNFRSF1B; TIMP-3; TIMP-2; TIMP-1;
Thrombospondin; TACE/ADAM17; Stabilin-1 RECK; Pref-1/DLK-1/FA1; PAR1;
Osteopontin; NCAM-L1; NCAM-1/CD56; MMP-2 (total); MD-1; LOX-1/SR-E1;
Lipocalin-2/NGAL; JAM-A; Integrin _6; Integrin _5; Integrin _4/CD104; Integrin
_3/CD61; Integrin _2/CD18; Integrin _1/CD29; IL-15 R_ ;IL-1RII; ICAM-2/CD102; HB-
EGF; Galectin-3BP/MAC-2BP; Galectin-3; Galectin-1; Epiregulin; Endoglin/CD105;
EMMPRIN/CD147; CXCL8/IL- 8; CX3CL1/Fractalkine; CEACAM-1/CD66a;
CD155/PVR; CD99; CD90/Thy1; CD58/LFA-3; CD44H; CD40; Ligand/TNFSF5;
CD36/SR/B3; CD31/PECAM-1; CD23;/Fc_RII CD9; C1qR1/CD93; BCAM; BACE-1
APP (pan); Amphiregulin; ALCAM/CD166; ADAM10 ; ADAM9; ACE

Accordingly, in some embodiments the presently disclosed subject matter provides specific inhibitors of ADAM8 activity and methods for using the same to study the biological functions of ADAM8 and/or for the treatment of diseases and disorders associated with undesirable ADAM8 biological activities.

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### 6.1. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the invention and the claims.

Following long-standing patent law convention, the terms “a”, “an”, and “the” refer to “one or more” when used in this application, including the claims. For example, the phrase “an antibody” refers to one or more antibodies, including a plurality of the same antibody. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and other inactive agents can and likely would be present in the pharmaceutical composition.

With respect to the terms “comprising”, “consisting of”, and “consisting essentially of”, where one of these three terms is used herein, the presently disclosed and claimed subject matter can include the use of either of the other two terms. For example, in some embodiments, the presently disclosed subject matter relates to compositions comprising antibodies. It would be understood by one of ordinary skill in the art after review of the instant disclosure that the presently disclosed subject matter thus encompasses compositions that consist essentially of the antibodies of the presently disclosed subject matter, as well as compositions that consist of the antibodies of the presently disclosed subject matter.

The term “subject” as used herein refers to a member of any invertebrate or vertebrate species. Accordingly, the term “subject” is intended to encompass any member of the Kingdom Animalia including, but not limited to the phylum Chordata (e.g., members of Classes Osteichythyes (bony fish), Amphibia (amphibians), Reptilia (reptiles), Ayes (birds), and Mammalia (mammals)), and all Orders and Families encompassed therein.

The compositions and methods of the presently disclosed subject matter are particularly useful for warm-blooded vertebrates. Thus, the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

Similarly, all genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes presented herein, the human amino acid sequences disclosed are intended to encompass homologous genes and gene products from other animals including, but not limited to other mammals, fish, amphibians, reptiles, and birds. Also encompassed are any and all nucleotide sequences that encode the disclosed amino acid sequences, including but not limited to those disclosed in the corresponding GENBANK® entries.

As used herein, the term “amino acid” encompasses any naturally occurring amino acid, modified forms thereof, and synthetic amino acids.

Naturally occurring, levorotatory (L-) amino acids are shown in Table 2.

TABLE 2
Naturally occurring amino acids

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Alternatively, the amino acid can be a modified amino acid residue (nonlimiting examples are shown in Table 3) and/or can be an amino acid that is modified by post-translational modification (e.g., acetylation, amidation, formylation, hydroxylation, methylation, phosphorylation or sulfatation). For example, the polypeptides disclosed herein may be glycosylated. For example, in expression eukaryotic cells N67 or N91 or another amino acid may have a carbohydrate moiety covalently attached. The glycosylation of various residues may occur as part of expression in a eukaryotic cell system or glycosylation sites may be specifically introduced or eliminated by mutation of asparagine to amino acid.

TABLE 3
Selected Modified Amino Acids

| Modified Amino Acid Residue | Abbreviation |
|---|---|
| Amino Acid Residue Derivatives | |
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Abe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,21-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | aIle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-C1) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

The terms “cancer” and “tumor” are used interchangeably herein and can refer to both primary and metastasized solid tumors and carcinomas of any tissue in a subject, including but not limited to blood, breast; colon; rectum; lung; oropharynx; hypopharynx; esophagus; stomach; pancreas; liver; gallbladder; bile ducts; small intestine; urinary tract including kidney, bladder, and urothelium; female genital tract including cervix, uterus, ovaries (e.g., choriocarcinoma and gestational trophoblastic disease); male genital tract including prostate, seminal vesicles, testes and germ cell tumors; endocrine glands including thyroid, adrenal, and pituitary; skin (e.g., hemangiomas and melanomas), bone or soft tissues; blood vessels (e.g., Kaposi's sarcoma); brain, nerves, eyes, and meninges (e.g., astrocytomas, gliomas, glioblastomas, retinoblastomas, neuromas, neuroblastomas, Schwannomas and meningiomas). As used herein, the terms “cancer and “tumor” are also intended to refer to multicellular tumors as well as individual neoplastic or pre-neoplastic cells. In some embodiments, a cancer or a tumor comprises a cancer or tumor of an epithelial tissue such as, but not limited to a carcinoma. In some embodiments, a tumor is an adenocarcinoma, which in some embodiments is an adenocarcinoma of the pancreas, breast, ovary, colon, or rectum, and/or a metastatic cell derived therefrom.

As used herein, the term “polypeptide” encompasses both peptides and proteins, unless indicated otherwise.

A “nucleic acid” or a “polynucleotide” is a sequence of nucleotide bases, and may be RNA, DNA or DNA-RNA hybrid sequences (including both naturally occurring and non-naturally occurring nucleotide), but in representative embodiments are either single or double stranded DNA sequences.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” nucleotide is enriched by at least about 10-fold, about 100-fold, about 1000-fold, about 10,000-fold or more as compared with the starting material.

According to a particular embodiment, the amino acid sequence of the ADAM8 prodomain peptide is preferably at least 80 , at least 85 , at least 87%, at least 89%, at least 91%, at least 93%, at least 95%, at least 97%, at least 99% or more say 100% homologous to the ADAM8 prodomain peptide sequence described in SEQ ID NO: 2-9 or 14-20 as determined using BlastP software of the National Center of Biotechnology Information (NCBI) using default parameters) and is capable of inhibiting an enzymatic activity of ADAM8, wherein the percent homology does not include the mutations described herein. In measuring homology between a peptide and a protein of greater size, homology is measured only in the corresponding region; that is, the protein is regarded as only having the same general length as the peptide, allowing for gaps and insertions.

ADAM8 prodomain nucleic acids include nucleic acids comprising the following polynucleotides: (1) all or a fragment of SEQ ID NOS: 21-26 contain the modifications described herein, wherein the fragment encodes an ADAM8 prodomain that can modulate the biological activity of ADAM8; (2) a polynucleotide including nucleotide sequences at least 80%. 85%, 90%. 95%, 97%, 98%, 99%, 99.5%, or 99.7% identical to SEQ ID NOS: 21-26 contain the modifications described herein wherein the alignment window is at least 100, 125, 150, 175, 200, 225, 250, 300, 400, 500, 600, 800, 1000, or 1200 nucleotides long and wherein the sequence encodes an protein that can modulate the biological activity of ADAM8; (3) a polynucleotide that comprises not more than 1, 2, 3, 4, 6. 8, 10, 15, 20, 25, or 30 alteration(s) of a single nucleotide relative to SEQ ID NOS: 21-26 with the modifications described herein, wherein an alteration can be an insertion, deletion or substitution of a single nucleotide, and wherein the polynucleotide encodes an ADAM8 prodomain can modulate the biological activity of ADAM8; and (4) a polynucleotide that encodes an ADAM8 prodomain as described herein, which includes fragments, derivatives and variants of an ADAM8 prodomain. In a one embodiment, the ADAM8 prodomain is produced by replacing the leader sequence with a heterologous secretion signal peptide.

Also included in this disclosure are ADAM8 prodomain nucleic acids which hybridize under moderate or high stringency conditions with all or a fragment of SEQ ID NOS: 21-26, wherein the fragment encodes an ADAM8 prodomain that can modulate the biological activity of ADAM8. Hybridization techniques are well known in the art and are described by Sambrook, J., E. F. Fritsch, and T. Maniatis (Molecular Cloning: A Laboratory Manual, Cold Spring Harbor Laboratory Press, Cold Spring Harbor, N.Y., chapters 9 and 11, (1989)) and Current Protocols in Molecular Biology (F. M. Ausubel et al., eds., John Wiley & Sons, Inc., sections 2.10 and 6.3-6.4 (1995)), the relevant portions of which are incorporated by reference herein. Moderately stringent conditions for filter hybridizations include hybridization in about 50% formamide, 6× SSC at a temperature from about 42 C to 55 C and washing at about 60 C in 0.5× SSC, 0.1% SDS. Highly stringent conditions are defined as hybridization conditions as above, but with washing at approximately 68 C in 0.2× SSC, 0.1% SDS. SSPE (1× SSPE is 0.15 M NaCI, 10 mM NaH₂P0₄, and 1.26 mM EDTA, pH 7.4) can be substituted for SSC (1× SSC is 0.15 M NaCI and 1 5 mM sodium citrate) in the hybridization and wash buffers; washes, optionally at least two washes, are performed for 15 minutes after hybridization is complete.

It should be understood that the wash temperature and wash salt concentration can be adjusted as necessary to achieve a desired degree of stringency by applying the basic principles that govern hybridization reactions and duplex stability, as known to those skilled in the art and described further below (see e.g., Sambrook et al., supra). When nucleic acids of known sequence are hybridized, the hybrid length can be determined by aligning the sequences of the nucleic acids (for example, using GAP) and identifying the region or regions of optimal sequence complementarity. The hybridization temperature for hybrids anticipated to be less than 50 base pairs in length should be 5 to 10° C. less than the melting temperature (Tm) of the hybrid, where Tm is determined according to the following equations. For hybrids less than 18 base pairs in length, Tm (degrees C.)=2(# of A+T bases)+4(# of G+C bases). For hybrids above 18 base pairs in length, Tm (degrees C.)=81.5+16.6(log₁₀[Na+])+0.41 (% G+C)−(600 N), where N is the number of bases in the hybrid, and [Na+] is the concentration of sodium ions in the hybridization buffer. Each such hybridizing nucleic acid has a length that is at least 15 nucleotides (or at least 18 nucleotides, or at least 20, or at least 25, or at least 30, or at least 40, or at least 50, or at least 100. Sambrook et al., supra.

An “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, about 100-fold, about 1000-fold, about 10,000-fold or more as compared with the starting material.

As used herein, by “isolate” or “purify” (or grammatical equivalents) a polypeptide or a virus vector, it is meant that the polypeptide or the virus vector is at least partially separated from at least some of the other components in the starting material. In representative embodiments an “isolated” or “purified” polypeptide or virus vector is enriched by at least about 10-fold, about 100-fold, about 1000-fold, about 10,000-fold or more as compared with the starting material.

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., anti-cancer effects or improvement in transplant survivability.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the disclosure. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the present disclosure.

“Effective amount” refers to an amount that, when administered to a subject for treating a disease, disorder or condition, is sufficient to affect or alleviate one or more symptoms of the disease, disorder, or condition. The “effective amount” may vary depending, for example, on the disease, disorder, or condition, and/or symptoms thereof, the severity of the disease, disorder, condition and/or symptoms thereof, the age, weight, and/or health of the subject, and the judgment of the prescribing physician. An appropriate amount in any given instance may be ascertained by those skilled in the art or capable of determination by routine experimentation. In some embodiments, the effective amount is a therapeutically effective amount.

The term “vector,” as used herein, means any nucleic acid entity capable of amplification in a host cell. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated. The choice of vector will often depend on the host cell into which it is to be introduced. Vectors include, but are not limited to plasmid vectors, phage vectors, viruses or cosmid vectors. Vectors usually contain a replication origin and at least one selectable gene, i.e., a gene which encodes a product which is readily detectable or the presence of which is essential for cell growth.

The term “vector” includes “virus vector,” or “gene delivery vector” includes to a virus (e.g., adenovirus vector (AV), adeno-associated vector (AAV), a herpes simplex vector (HSV), or a retrovirus vector) particle that functions as a nucleic acid delivery vehicle, and which comprises the vector genome (e.g., viral DNA [vDNA]) packaged within a virion. Alternatively, in some contexts, the term “virus vector” may be used to refer to the viral vector genome/vDNA alone.

The term “gene therapy” refers to a method of changing the expression of an endogenous gene by exogenous administration of a gene. As used herein, “gene therapy” also refers to the replacement of defective gene encoding a defective protein, or replacement of a missing gene, by introducing a functional gene corresponding to the defective or missing gene into somatic or stem cells of an individual in need. Gene therapy can be accomplished by ex vivo methods, in which differentiated or somatic stem cells are removed from the individual's body followed by the introduction of a normal copy of the defective gene into the explanted cells using a viral vector as the gene delivery vehicle. In addition, in vivo direct gene transfer technologies allow for gene transfer into cells in the individual in situ using a broad range of viral vectors, liposomes, protein DNA complexes or naked DNA in order to achieve a therapeutic outcome. The term “gene therapy” also refers to the replacement of a defective gene encoding a defective protein by introducing a polynucleotide that functions substantially the same as the defective gene or protein should function if it were not defective into somatic or stem cells of an individual in need. Gene therapy methods may be found in Amer (2014) “Gene therapy for cancer: present status and future perspective” Mol. Cell. Therapies 2:27 p1-19 or Goswami et al. (2019) “Gene Therapy Leaves a Vicious Cycle” Front in Oncology pub 24 April 2019, p1-25. Additional examples of gene therapy including viral gene delivery systems are adenovirus vectors (AV) WO2000/029600 (Georgetown Univ. & Synergene, Inc.); adeno-associated virus (AAV) WO2019/011893, WO2019/016349, WO2019/122293, WO2019/129859 (all to Uniqure IP BV); lipid complexes WO2017/186711 (Invivogen); targeted liposomes WO2000/050008 (Georgetown Univ. & Synergene, Inc.).

The term “gene editing” refers to the insertion, deletion, or replacement of DNA at a specific site in the genome of an organism or cell. Gene editing may be performed using one or more targeted nuclease systems, such as a CRISPR/Cas system, a Zn finger nuclease, a TALEN, a homing endonuclease, etc. For an example of a zinc finger nuclease, see WO2017/223107. In one embodiment, the gene editing may be used in conjunction with preparing chimeric antigen receptor T-cells (CAR-T cells), see WO2017/079400 (Univ. of Pennsylvania) or US Pat. No. 10,415,016 (Poseida Therapeutics, Inc.). Various methods and compositions for targeted cleavage of genomic DNA have been described. Also See, e.g., US Pat. Nos. 6,503,717; 6,534,261; 6,599,692; 6,689,558; 7,067,317; 7,262,054; 7,888,121; 7,972,854; 7,914,796; 7,951,925; 8,034,598; 8,110,379; 8,409,861; 8,586,526; 8,623,618; and 10,407,476; US Pat. Pub. Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2015/0056705.

### 6.2. Modified ADAM8 Prodomain Peptides

The presently disclosed subject matter provides in some embodiments modified ADAM8 prodomain peptides that, as compared to their unmodified counterparts, have desirable biological activities. By way of example and not limitation, the modified ADAM8 prodomain peptides of the presently disclosed subject matter can be in some embodiments more or less sensitive to cleavage by furin and furin-like proteins, in some embodiments more or less sensitive to cleavage by autocatalysis, and in some embodiments more or less likely to form dimers and other multimers via cysteine crosslinking, either to themselves or other members of the ADAM family of proteins (including but not limited to ADAM8), and in some embodiments.

As used herein, the phrases “modified ADAM8 prodomain peptides”, “modified ADAM8 peptides”, “the ADAM8 modulating peptides”, and the like refer to peptides that have one or more modifications of the amino acid sequences set forth in SEQ ID NO: 2-9 or 14-20 and/or other modifications that result in some desirable biological or biochemical property of a modified ADAM8 peptide as compared to an ADAM8 peptide that does not have the modification(s), including but not limited to a wild type ADAM8 prodomain peptide upon which it is based. By way of example and not limitation, the types of modifications that can be introduced into the modified ADAM8 peptides of the presently disclosed subject matter include amino acid substitutions, deletions, additions, and other types of modifications designed to alter one or more biological or biochemical properties of a modified ADAM8 peptide such as but not limited to solubility in a given solvent (including but not limited to a biological fluid such as blood, serum, cerebrospinal fluid, etc.), a dissociation constant with respect to a binding partner, a half maximal inhibitory concentration (IC50) towards an enzyme (such as but not limited to ADAM8 or another member of the ADAM family of disintegrins and metalloproteinases), and/or to improve various pharmokinetic properties of interest.

With respect to the modifications that are encompassed within the presently disclosed subject matter, there are at least three biologically relevant features of the members of the ADAM family such as ADAM8 that can be exploited to produce the modified ADAM8 peptides of the presently disclosed subject matter. By way of example and not limitation, ADAM8 proteins and prodomain peptides derived therefrom are characterized by recognition sites for furin and furin-like endoproteases, recognitions sites for autocatalyis, and numerous cysteine residues that can form homo- and heterodimers and higher order multimers via formation of disulfide bonds. Cleavage by furin and furin-like endoproteases and autocatalysis as well as disulfide bond formation is relevant to several of the biological activities of the members of the ADAM family such as ADAM8, and the amino acid sequences responsible for these activities can be modified in order to modulate the biological activities of the members of the ADAM family such as ADAM8.

As used throughout the instant disclosure, references to amino acid positions in, for example, SEQ ID NOS: 1, 12, OR 13, also relate to the corresponding amino acid positions in any of SEQ ID NOs: 2-9 or 14-20. As such, by way of example and not limitation, amino acid positions 35, 41, 43, 44, 105, 124, 152-164, and 167 of SEQ ID NOS: 1, 12, OR 13 correspond to amino acid positions 20, 26, 28, 29, 90, 109, 137-149, and 152 of SEQ ID NOs: 2-4 and 6-20, and amino acid positions 20, 26, 28, 29, 89, 108, 136-148, and 151 of SEQ ID NOs: 5).

The wild type human ADAM8 amino acid sequence is represented by SEQ ID NO: 1, which corresponds to Accession No. NP_001100.3 of the GENBANK® biosequence database (encoded by Accession No. NM_001109.5 of the GENBANK® biosequence database). An exemplary prodomain peptide is represented by SEQ ID NOS: 1, OR 13 and the human prodomain represented by SEQ ID NO: 2, has a furin recognition site at amino acids 41-44 of SEQ ID NOS: 1 OR 13. As disclosed herein, modifications of amino acids 41, 43, and/or 44 of SEQ ID NOS: 1 OR 13 result in modified ADAM8 peptides that have greater or lesser sensitivities to cleavage by furin and furin-like endoproteases. Similarly, the wild type human ADAM8 prodomain peptide represented by SEQ ID NO: 2 has possible autocatalysis sites between amino acids 137-149 of SEQ ID NO: 2. Also similarly, modifications of any of the cysteines of SEQ ID NO: 2 that are involved with disulfide bond formation, including but not limited to cysteines 105, 124, and 167 of SEQ ID NOS: 1 OR 13, result in modified ADAM8 peptides that have greater or lesser ability to form disulfide bonds with ADAM8 proteins and/or other ADAM family member proteins. As would also be understood by one of ordinary skill in the art, combinations of furin site modifications, autocatalytic site modifications, and/or cysteines can result in modified ADAM8 peptides that have multiple new functionalities, as desired.

Therefore, in some embodiments, the ADAM8 modulating peptides of the presently disclosed subject matter peptides with amino acid sequences derived from SEQ ID NO: 2 that include at least one amino acid substitution or other modification of at least one of amino acids 41, 43, and/or 44 of SEQ ID NOS: 1 OR 13 (i.e., the furin recognition sequence), at least one of amino acids 152-164 of SEQ ID NOS: 1 OR 13 (i.e., the autocatalytic site), and/or at least one of cysteines 105, 124, and 167 of SEQ ID NOS: 1 or 13. It is understood that modifications such as but not limited to amino acid substitutions can occur at any one or more of amino acids 152-164 of SEQ ID NOs: 1 (corresponds to amino acids 137-149 of SEQ ID NO: 2, 3, 18, 19, or 20), in any combination, and all combinations and subcombinations of amino acid substitutions at any combination or subcombination of these amino acid positions are encompassed by the presently disclosed subject matter.

In addition to and/or alternatively to any of the autocatalytic and/or furin modifications described herein above, one or more of the cysteines involved in disulfide bond formation can be modified. In some embodiments, these cysteines include cysteines 105, 124, and/or 167 of SEQ ID NOS: 1, 12, or 13 (sites 90, 109, and/or 152 of SEQ ID NOS: 2, 3, 16, 18-20) in any combination or subcombination. Thus, and with reference to the amino acid positions of SEQ ID NOS: 1, 12, or 13, in some embodiments the ADAM8 prodomain peptides of the presently disclosed subject matter include a modification of cysteine 105, a modification of cysteine 124, a modification of cysteine 167, modifications of both cysteines 105 and 124, modifications of both cysteines 124 and 167, or modifications of all of cysteines 105, 124, and 167. In some embodiments and with reference to the amino acid positions of SEQ ID NOS: 1, 12, or 13, the modifications at these cysteines are substitutions of any amino acid, with each of cysteines 105, 124, and 167 being independently substituted with the same or a different amino acid. In some embodiments and with reference to the amino acid positions of SEQ ID NOS: 1, 12, or 13, cysteines 105, 124, and/or 167are independently substituted with serine. In some embodiments the cysteines are chemically reacted for a sulfhydryl modifying agent such as but not limited to a maleimide ester.

In addition to the modifications at positions 41, 43, 44, 105, 124, 152-164, and 167 of SEQ ID NOS: 1, 12, or 13 and the corresponding positions in any of SEQ ID NOs: 2-9 or 14-20, other modifications of the amino acid sequence can be introduced. By way of example and not limitation, one or more amino acids (in some embodiments, 1, 2, 3, 4, 5, or more amino acids) can be added to the N-terminus and/or the C-terminus of a peptide of any of SEQ ID NOs: 2-9 or 14-20. By way of additional example and not limitation, a cysteine residue alone or in combination with other amino acids can be added to the N-terminus and/or the C-terminus to provide a functionality known to be possessed by cysteine residues. For example, a cysteine residue alone or in combination with other amino acids can be added to the N-terminus and/or the C-terminus to provide a site for pegylation of an ADAM8 modulating peptide.

Modifications and changes can be made in the structure of an ADAM8 modulating peptides of the presently disclosed subject matter and still obtain a molecule having ADAM8 modulating properties. For example, certain amino acids can be substituted for other amino acids in a sequence without appreciable loss of peptide activity. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide with ADAM8 modulating properties.

In making such changes, the hydropathic index of amino acids can be considered. The importance of the hydropathic amino acid index in conferring interactive biologic function on a polypeptide is generally understood in the art (Kyte & Doolittle, 1982). It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within±2 is preferred, those which are within±1 are particularly preferred, and those within±0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated herein by reference, states that the greatest local average hydrophilicity of a polypeptide, as governed by the hydrophilicity of its adjacent amino acids, correlates with its immunogenicity and antigenicity: i.e., with a biological property of the polypeptide. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within±2 is preferred, those which are within±1 are particularly preferred, and those within±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine (see Table 4, below). The presently disclosed subject matter thus contemplates functional or biological equivalents of a peptide as set forth above.

TABLE 4
Exemplary Amino Acid Substitutions

| Original Residue | Exemplary Substitutions | Original Residue | Exemplary Substitutions |
|---|---|---|---|
| Ala | Gly; Ser | Ile | Leu; Val |
| Arg | Lys | Leu | Ile; Val |
| Asn | Gln; His | Lys | Arg |
| Asp | Glu | Met | Leu; Tyr |
| Cys | Ser | Ser | Thr |
| Gln | Asn | Thr | Ser |
| Glu | Asp | Trp | Tyr |
| Gly | Ala | Tyr | Trp; Phe |
| His | Asn; Gln | Val | Ile; Leu |

Biological or functional equivalents of a peptide or polypeptide can be prepared using site-specific mutagenesis according to procedures well known in the art. Accordingly, amino acid residues can be added to or deleted from the ADAM8 modulating peptides of the presently disclosed subject matter through the use of standard molecular biological techniques without altering the functionality of the peptide. Specific examples include the human ADAM8 prodomain peptides of the presently disclosed subject matter.

In some embodiments, the ADAM8 prodomain peptide of the presently disclosed subject matter consists of, consists essentially of, or comprises an amino acid sequence as set forth in any of SEQ ID NOs: 2-9 or 14-20 or is an ADAM8 prodomain peptide having at least 87%, 88%, 89%, 90%, 91%, 92%, 93%, 94%, 95%, 96%, 97%, 98%, 99%, or 100% homology to any one of SEQ ID NOs 3-9 or 14-20. In some embodiments, the ADAM8 prodomain peptide comprises one of SEQ ID NOs 3-9 or 14-20 or is an ADAM8 modulating peptide having at least 87%, 88%, 89%, 90%, 91%, 92%, 93%, 94%, 95%, 96%, 97%, 98%, 99%, or 100% homology to one of SEQ ID NOs 2-9 or 14-20 and is pegylated.

In view of the above, exemplary modified ADAM8 peptides of the presently disclosed subject matter can comprise, consist essentially of, and/or consist of the amino acid sequences set forth in any of SEQ ID NOs 2-9 or 14-20. SEQ ID NOS: 3, 19, and 20 are a general consensus sequence, wherein the positions that correspond to amino acid positions 41, 43, 44, 67, 91 105, 124, 152-164, and 167 of SEQ ID NOS: 1, 12, or 13 can be substituted with any amino acid, either individually or in any combination, as set forth herein.

### 6.3. Expression Systems and Modified ADAM8 Prodomains

In some embodiments an ADAM8 modulating peptide can be conjugated to a PEG group of about 1-40 kDa, or can be conjugated to a polymer, sugar residue, peptide, protein, antibody, antibody fragment, or other scaffold, a toxin, fluorophore or radionuclide. In some embodiments by adding a peptide sequence to the N- and/or the C-terminus that includes a cysteine to which a peg group, polymer, sugar residue, peptide, protein, antibody, antibody fragment, or other scaffold, a toxin, fluorophore or radionuclide can be attached. The ADAM8 modulating peptides of the presently disclosed subject matter can include peptides containing additional sequences (e g , amino acids) on the N- and/or the C-terminus that are necessary for successful expression of the ADAM8 modulating peptides in E. coli, insect cells, mammalian systems, etc. In addition, one or more tags can be added which aid in the purification of the ADAM8 modulating peptides of the presently disclosed subject matter. The tags can include, but are not limited to, His tags, C-myc tags, Flag tags, HA tags, Streptavidin tags, disulfide tags, and biotin tags. The sequences between the tag(s) and the prodomain peptides can in some embodiments comprise protease cleavage sites, such as those found for enterokinase, thrombin, and/or Tev proteases. The ADAM8 modulating peptides of the presently disclosed subject matter may include modifications that stabilize the peptide for in vivo use. Such modifications are generally known to those of skill in the art and include, but are not limited to, modification with fatty acids and pegylation, incorporation of D amino acids, and substitution, deletion, and/or addition of amino acids.

ADAM8 prodomain peptides can be made as follows. A nucleic acid that encodes an ADAM8 prodomain peptide, as described herein, can be introduced into a vector, which can be introduced into a host cell. Vectors and host cells comprising nucleic acids encoding an ADAM8 prodomain peptide are encompassed by the invention. The host cell containing the nucleic acids encoding an ADAM8 prodomain peptide can be cultured under conditions such that the ADAM8 prodomain peptide can be expressed. The expressed ADAM8 prodomain peptide can then be obtained from the medium in which the cells are cultured or from the cells and purified by any of the many appropriate means known in the art. In addition, genetic engineering methods for the production of ADAM8 prodomain peptides include the expression of the polynucleotide molecules in cell free expression systems, in cellular hosts, in tissues, and in animal models, according to known methods.

The vector can include a selectable marker and an origin of replication, for propagation in a host. The vector can further include suitable transcriptional or trans lational regulatory sequences, such as those derived from mammalian, microbial, viral, or insect genes, operably linked to the nucleic acid encoding the ADAM8 prodomain peptide. Examples of such regulatory sequences include transcriptional promoters, operators, or enhancers, mRNA ribosomal binding sites, and appropriate sequences that control transcription and translation. Nucleotide sequences are operably linked when the regulatory sequence functionally relates to the DNA encoding the target protein. Thus, a promoter nucleotide sequence is operably linked to an %%% nucleic sequence if the promoter nucleotide sequence directs the transcription of the ADAM8 prodomain peptide-encoding sequence. If the ADAM8 prodomain peptide is a fusion protein, a nucleic acid sequence encoding a portion of the fusion protein, for example, a signal sequence, can be part of a vector, and a nucleic acid encoding an ADAM8 prodomain peptide can be inserted into the vector such that a protein comprising the added signal sequence plus the ADAM8 prodomain peptide is encoded by the vector.

Suitable host cells for expression of ADAM8 prodomain peptides include prokaryotic cells, yeast cells, plant cells, insect cells, and higher eukaryotic cells. The regulatory sequences in the vector will be chosen such that they are operable in the host cell. Suitable prokaryotic host cells include bacteria of the genera Escherichia, Bacillus, and Salmonella, as well as members of the genera Pseudomonas, Streptomyces, and Staphylococcus. For expression in prokaryotic cells, for example, in E. coli the polynucleotide molecule encoding an ADAM8 prodomain peptide preferably includes an N-terminal methionine residue to facilitate expression of the recombinant polypeptide. The N-terminal methionine may optionally be cleaved from the expressed polypeptide. Suitable yeast host cells include cells from genera including Saccharomyces, Pichia, and Kluveromyces. Preferred yeast hosts are S. cerevisiae and P. pastoris. A suitable system for expression in an insect host cell is described, for example, in the review by Luckow and Summers (1988 BioTechnology 6 47-55), the relevant portions of which are incorporated herein by reference. Suitable mammalian host cells include the COS-7 line of monkey kidney cells (Gluzman et al. 1981 Cell 23 175-182), baby hamster kidney (BHK) cells, Chinese hamster ovary (CHO) cells (Puck et al. 1958 PNAS USA 60 1275-1281), CV-1 (Fischer et al. 1970 Int J Cancer 5 21-27), 293 cells from human kidney (American Type Culture Collection (ATCC®) catalog no. CRL-10852™), and human cervical carcinoma cells (HELA) (ATCC® CCL 2). The relevant portions of the references referred to in this paragraph are incorporated herein by reference.

Expression vectors for use in cellular hosts generally comprise one or more phenotypic selectable marker genes. Such genes encode, for example, a protein that confers antibiotic resistance or that supplies an auxotrophic requirement. A wide variety of such vectors are readily available from commercial sources. Examples include pGEM vectors (Promega), pSPORT vectors, and pPROEX vectors (InVitrogen, Life Technologies, Carlsbad, Calif.), Bluescript vectors (Stratagene), and pQE vectors (Qiagen). Yeast vectors will often contain an origin of replication sequence from a yeast plasmid, an autonomously replicating sequence (ARS), a promoter region, sequences for polyadenylation, sequences for transcription termination, and a selectable marker gene. Vectors replicable in both yeast and E. colt (termed shuttle vectors) may also be used. In addition to the above-mentioned features of yeast vectors, a shuttle vector will also include sequences for replication and selection in E. colt. Direct secretion of the target polypeptides expressed in yeast hosts may be accomplished by the inclusion of nucleotide sequence encoding the yeast a-factor leader sequence at the 5′ end of the %%%-encoding nucleotide sequence. Brake 1989 Biotechnology 13 269-280.

Examples of suitable expression vectors for use in mammalian host cells include pcD A3.1/Hygro (Invitrogen), pDC409 (McMahan et al. 1991 EMBO J 10: 2821-2832), and pSVL (Pharmacia Biotech). Expression vectors for use in mammalian host cells can include transcriptional and translational control sequences derived from viral genomes.

Commonly used promoter sequences and enhancer sequences that can be used to express %%% RNA include, but are not limited to, those derived from human cytomegalovirus (CMV). Adenovirus 2, Polyomavirus, and Simian virus 40 (SV40). Methods for the construction of mammalian expression vectors are disclosed, for example, in Okayama and Berg (1982 Mol Cell Biol 2: 161-170), Cosman et al. (1986) Mol Immunol 23:935-941), Cosman et al. (1984 Nature 3 12: 768-771), EP-A-0367566, and WO 91/18982. The relevant portions of these references are incorporated herein by reference.

### 6.4. Coupling/Purification Tags

A wide variety of appropriate coupling methods may be used to attach a peptide to a solid support for purification, diagnostics, etc. The coupling may be performed with covalent linkages such as amide linkages (e g , amino NHS-ester), ester bonds, phosphoester bonds, or disulfide bonds. The coupling may also be performed using methods such as affinity tags, such as antigenic tags or other binding methods (e.g., antibody-protein A; biotin-streptavidin; FLAG-tag (Sigma-Aldrich, Hopp et al. 1988 Nat Biotech 6:1204-1210); glutathione S-transferase (GST)/glutathione; hemagluttanin (HA) (Wilson et al., 1984 Cell 37:767); intein fusion expression systems (New England Biolabs, USA) Chong et al. 1997 Gene 192(2), 271-281; maltose-binding protein (MBP)); poly His-(Ni or Co) (Gentz et al., 1989 PNAS USA 86:821-824); or thiol-gold. Fusion proteins containing GST-tags at the N-terminus of the protein are also described in U.S. Pat. No. 5,654,176 (Smith). Magnetic separation techniques may also be used such as Strepavidin-DynaBeads® (Life Technologies, USA). Alternatively, photo-cleavable linkers may be used, e.g., U.S. Pat. No. 7,595,198 (Olejnik & Rothchild). A wide variety of coupling methods, including polystyrene affinity peptides, are reviewed by Nakanishi et al. Nakanishi et al. 2008 Curr Proteomics 5 161-175.

### 6.5. Labeled Peptides

The invention also includes all suitable isotopic variations incorporated into the ADAM8 prodomains described herein. An isotopic variation is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulfur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Substitution with positron emitting isotopes, such as ¹¹C, ¹⁸F, ¹⁵O and ¹³N, can be useful in Positron Emission Topography (PET) studies.

Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents. In another embodiment, the isotope-labeled compounds contain deuterium (²H), tritium (³H) or ¹⁴C isotopes. Isotope-labeled compounds of this invention can be prepared by the general methods well known to persons having ordinary skill in the art.

Such isotope-labeled compounds can be conveniently prepared by carrying out the procedures disclosed in the Examples disclosed herein and Schemes by substituting a readily available isotope-labeled reagent for a non-labeled reagent. In some instances, compounds may be treated with isotope-labeled reagents to exchange a normal atom with its isotope, for example, hydrogen for deuterium can be exchanged by the action of a deuteric acid such as D₂SO₄/D₂O. Alternatively, deuterium may be also incorporated into a compound using methods such as through reduction such as using LiAlD₄ or NaBD₃, catalytic hydrogenation or acidic or basic isotopic exchange using appropriate deuterated reagents such as deuterides, D₂ and D₂O. In addition to the above, PCT publications, WO2014/169280; WO2015/058067; U.S. Pat. Nos. 8,354,557; 8,704,001 and US Patent Application Publication Nos.; 2010/0331540; 2014/0081019; 2014/0341994; 2015/0299166.

In some embodiments, the presently disclosed subject matter provides pharmaceutical compositions comprising an ADAM8 modulating peptide and a physiologically acceptable carrier. In some embodiments, the pharmaceutical composition comprises an ADAM8 modulating peptide consisting or, consisting essentially of, and/or comprising the amino acid sequence set forth in SEQ ID NOs 3-9 or 14-20.

### 6.6. Pharmaceutical Compositions

A composition of the presently disclosed subject matter is typically administered parenterally in dosage unit formulations containing standard, well-known nontoxic physiologically acceptable carriers, adjuvants, and vehicles as desired. The term parenteral as used herein includes intravenous, intramuscular, intraarterial injection, intraperitoneal, topical, or infusion techniques.

Injectable preparations, for example sterile injectable aqueous or oleaginous suspensions, are formulated according to known techniques using suitable dispersing or wetting agents and suspending agents. The sterile injectable preparations can also be sterile injectable solutions and/or suspensions in a nontoxic parenterally acceptable diluent or solvent, for example, as a solution in 1,3-butanediol.

Exemplary, non-limiting acceptable vehicles and solvents that may be employed are water, Ringer's solution, and isotonic sodium chloride solution. In addition, sterile, fixed oils are conventionally employed as solvents or suspending media. For this purpose, any bland fixed oil can be employed including synthetic mono- or di-glycerides. In addition, fatty acids such as oleic acid find use in the preparation of injectables. Preferred carriers include neutral saline solutions buffered with phosphate, lactate, Tris, and the like.

Any of the above-described therapeutic agents can be administered in the form of a composition, that is, with one or more additional components such as a physiologically acceptable carrier, excipient, or diluent. For example, a composition may comprise a soluble ADAM8 prodomain protein as described herein plus a buffer, an antioxidant such as ascorbic acid, a low molecular weight polypeptide (such as those having less than 10 amino acids), a protein, amino acids, carbohydrates such as glucose, sucrose, or dextrin, chelating agent such as EDTA, glutathione, and/or other stabilizers, excipients, and/or preservatives. The composition may be formulated as a liquid or a lyophilizate. Further examples of components that may be employed in pharmaceutical formulations are presented in Remington's Pharmaceutical Sciences, 16^{th} Ed., Mack Publishing Company, Easton, Pa., (1980), the relevant portions of which are incorporated herein by reference.

Compositions comprising therapeutic molecules described above can be administered by any appropriate means including, but not limited to, parenteral, topical, oral, nasal, vaginal, rectal, or pulmonary (by inhalation) administration. If injected, the composition(s) can be administered intra-articularly, intravenously, intraarterially, intramuscularly, intraperitoneally or subcutaneously by bolus injection or continuous infusion. Localized administration, that is, at the site of disease, is contemplated, as are transdermal delivery and sustained release from implants, skin patches, or suppositories. Delivery by inhalation includes, for example, nasal or oral inhalation, use of a nebulizer, inhalation in aerosol form, and the like. Administration via a suppository inserted into a body cavity can be accomplished, for example, by inserting a solid form of the composition in a chosen body cavity and allowing it to dissolve. Other alternatives include eye drops, oral preparations such as pills, lozenges, syrups, and chewing gum, and topical preparations such as lotions, gels, sprays, and ointments. In most cases, therapeutic molecules that are polypeptides can be administered topically or by injection or inhalation.

The therapeutic molecules described above can be administered at any dosage, frequency, and duration that can be effective to treat the condition being treated. The dosage depends on the molecular nature of the therapeutic molecule and the nature of the disorder being treated. Treatment may be continued as long as necessary to achieve the desired results. The periodicity of treatment may or may not be constant throughout the duration of the treatment. For example, treatment may initially occur at weekly intervals and later occur every other week. Treatments having durations of days, weeks, months, or years are encompassed by the invention. Treatment may be discontinued and then restarted.

Maintenance doses may be administered after an initial treatment. Dosage may be measured as milligrams per kilogram of body weight (mg/kg) or as milligrams per square meter of skin surface (mg/m²) or as a fixed dose, irrespective of height or weight. These are standard dosage units in the art. A person's skin surface area is calculated from her height and weight using a standard formula. For example, a therapeutic %%% protein can be administered at a dose of from about 0.05 mg kg to about 10 mg/kg or from about 0.1 mg/kg to about 1 .0 mg kg. Alternatively, a dose of from about 1 mg to about 500 mg can be administered. Or a dose of about 5 mg, 10 mg. 15 mg 20 mg, 25 mg, 30 mg. 35 mg, 40, mg, 45, mg, 50 mg, 55 mg, 60 mg, 100 mg, 200 mg, or 300 mg can be administered.

### 6.7. Methods for Use and Uses of the Modified ADAM8 Prodomain Peptides and Nucleic Acids Encoding such Peptides

The ADAM8 modulating peptides of the presently disclosed subject matter and compositions comprising or consisting essentially thereof are useful for modulating ADAM8 protein activity in vitro and in vivo. In some embodiments, the presently disclosed subject matter provides a method for modulating ADAM8 activity in vitro comprising contacting an ADAM8 modulating prodomain peptide with a solution or a cell comprising an ADAM8 protein under conditions and in an amount sufficient (referred to herein as “an effective amount” or, in the case of a treatment or preventive method, a “therapeutically effective amount”) to modulate the activity of the ADAM8 protein.

In some embodiments, the presently disclosed subject matter provides a method for modulating ADAM8 activity in vivo, the method comprising administering to a subject a composition (in some embodiments a pharmaceutical composition) comprising an ADAM8 modulating prodomain peptide in a therapeutically effective amount and via a route of administration sufficient to modulate ADAM8 activity. In some embodiments, the subject has a disease or disorder characterized at least in part by the presence of an excess of an ADAM8 biological activity (i.e., an undesirable level of an ADAM8 biological activity). In some embodiments, the disorder is characterized by one or more of inflammation, an allergic response, asthma, angiogenesis, vascular disease, cancer, a predisposition thereto, and/or a symptom or consequence thereof. In some embodiments, the disorder is a cancer and the symptom or consequence thereof comprises fibrosis.

Thus, in some embodiments the disease or disorder is characterized by one or more of Alzheimer's disease, cancer, inflammation, asthma, allergies, lung injuries, diseases related to excessive osteoclast formation or activity, vascular diseases, or a predisposition thereto, and/or a symptom or consequence thereof.

In some embodiments, the virus vector is a recombinant virus vector comprising a heterologous nucleic acid encoding a polypeptide or functional RNA of interest. In some embodiments, the nucleic acid is a nucleic acid encoding a polypeptide, including therapeutic (e.g., for medical or veterinary uses) or immunogenic (e.g., for vaccines) polypeptide or RNA.

It will be understood by those skilled in the art that the heterologous nucleic acid can be operably associated with appropriate control sequences. For example, the heterologous nucleic acid can be operably associated with expression control elements, such as transcription/translation control signals, origins of replication, polyadenylation signals, internal ribosome entry sites (IRES), promoters, and/or enhancers, and the like. Further, regulated expression of the heterologous nucleic acid(s) of interest can be achieved at the post-transcriptional level, e.g., by regulating selective splicing of different introns by the presence or absence of an oligonucleotide, small molecule and/or other compound that selectively blocks splicing activity at specific sites.

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the heterologous nucleic acid sequence. The promoter/enhancer element is generally chosen so that it functions in the target cell(s) of interest. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the heterologous nucleic acid sequence(s). Inducible promoters/enhancer elements for gene delivery can be tissue-specific or -preferred promoter/enhancer elements, and include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

The vectors according to the present disclosure provide a means for delivering heterologous nucleic acids into a broad range of cells, including dividing and non-dividing cells. The vectors can be employed to deliver a nucleic acid of interest to a cell in vitro, e.g., to produce a polypeptide in vitro or for ex vivo gene therapy. The virus vectors are additionally useful in a method of delivering a nucleic acid to a subject in need thereof e.g., to express an immunogenic or therapeutic polypeptide or a functional RNA. In this manner, the polypeptide or functional RNA can be produced in vivo in the subject. Further, the method can be practiced because the production of the polypeptide or functional RNA in the subject may impart some beneficial effect.

The vectors of the present disclosure can be employed to deliver a heterologous nucleic acid encoding a polypeptide or functional RNA to treat and/or prevent any disease state for which it is beneficial to deliver a therapeutic polypeptide.

Alternatively, the virus vector may be administered to a cell ex vivo and the altered cell is administered to the subject. The virus vector comprising the heterologous nucleic acid is introduced into the cell, and the cell is administered to the subject, where the heterologous nucleic acid encoding the immunogen can be expressed and induce an immune response in the subject against the immunogen. In particular embodiments, the cell is an antigen-presenting cell (e.g., a dendritic cell).

### 6.8. Kits

Another aspect of the present disclosure provides a kit for the inhibition of ADAM8 in connection with a disease or disorder described herein. The kit may comprise, consist of, or consist essentially of any one of the compositions described herein (whether peptide compositions, pegylated peptide compositions, or nucleic acids encoding the peptide compositions), a means of administering the composition, and instructions for use.

### 7. EXAMPLES

The following EXAMPLES provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### 7.1. Example 1

The Human Prodomain Peptides of ADAM8 Inhibits ADAM8 In Vitro

An ADAM8 prodomain peptide was prepared by cloning nucleic acid sequences optimal for bacterial expression along with a His₆ tag into a plasmid expression vector. Some prodomain peptides were prepared by transformation of BL21DE3 cells followed by expression at 37° C. which yielded inclusion bodies. SEQ ID NOS: 19-24 are examples of optimized DNA sequences that were used in this and subsequent experiments.

The inclusion bodies were prepared by breaking open the bacteria with 20 mM Tris, pH8, 1 mg/ml lysozyme (Sigma-Aldrich Corp., St. Louis, Missouri, United States of America), benzonase, (Sigma-Aldrich) protease inhibitors (Gold Biotechnology, Inc., St. Louis, Mo., United States of America) and 1× CelLytic™ solution (Sigma-Aldrich) for 40-60 minutes at room temperature and then the material was centrifuged for 30 minutes at 3,000 rpm. The pellet was then resuspended in the lysis buffer and rocked for 30 minutes at room temperature before spinning again to pellet the inclusion bodies. The inclusion bodies were solubilized with 8M urea, 20 mM Tris, pH9 with or without 2-4 mM TCEP, and then rocked with Ni-NTA beads from Fisher Scientific (Waltham, Mass., United States of America). Beads were washed with several volumes of 8 M urea, 20 mM Tris, with or without TCEP and then the material was eluted in 4-6 M urea and 20 mM Tris pH 9 with or without 2-4 mM TCEP and 0.66-1M imidazole pH 9. The prodomains were refolded from urea and then dialyzed or concentrated, diluted with 20 mM Tris, pH 8, and re-concentrated (repeated 3-4 times) to remove the remaining urea and imidazole using 20 mM Tris buffer pH 8. Prodomains were incubated at varying concentrations with human ADAM8 in 20 mM Tris pH 8 and 0.001% Brij® 35 brand detergent and 10 mM CaCl₂ in a black coated 96-well plate with 10-20 μM PEPDAB064, or PEPDAB013, fluorescence energy transfer substrates for ADAM family members (BioZyme Inc, Apex, North Carolina, United States of America). Data was fit to determine IC₅₀ values using Prism software. The results are presented in Table 5.

TABLE 5
IC50 values for Inhibition of ADAM8

| Construct | IC50 (uM) |
|---|---|
| SEQ ID NO: 4 | 40% inhibition at 5 uM |
| SEQ ID NO: 14 | 1.4 ± 1.2 uM |
| | 2.3 ± 0.3 uM |
| SEQ ID NO: 2 | 2.8 ± 1.2 uM |
| | 2.3 ± 0.8 uM |
| SEQ ID NO: 15 | 45% inhibition at 4.8 uM |
| SEQ ID NO: 16 | 0.4 uM |
| SEQ ID NO: 17 | 51% inhibition at 4.8 uM |
| SEQ ID NO: 15 (pegylated | 3.0 ± 2.3 uM |
| at C-terminus) | 1.8 ± 0.4 uM |
| SEQ ID NO: 14 (pegylated | 2.7 ± 0.5 uM |
| on 3 cysteines) | 2.3 ± 0.3 uM |
| SEQ ID NO: 17 (Pegylated | 24 ± 0.5 uM |
| on N-terminus) | |

### 7.2. Example 2

Selectivity Profile

For determination of specificity, SEQ ID NO:14 and pegylated SEQ ID NO:15 were incubated with either ADAM10 or ADAM17 (R & D Systems, Minneapolis, Minn., United States of America) with the same substrate buffer mix (at 2 μM or higher concentrations) as described for ADAM8 using PEPDAB013 (BioZyme Inc, Apex, N.C., United States of America). There was no inhibition of either enzyme.

### 7.3. Example 3

Dimerization and Multimerization of Prodomain Due to Oxidation

The wild type prodomain peptide (SEQ ID NO: 2 and SEQ ID NO:14) have three cysteines. Upon incubation in the cold, or at higher temperatures, or upon storage frozen at −60° C., the protein sulfhydryl groups can react with one another upon oxidation to form disulfide bonds. When the prodomains are stored for extended periods of time, oxidation occurs, and sometimes the prodomains precipitate. FIG. 5 shows the dimerized prodomain of SEQ ID NO:14 (FIG. 5).

### 7.4. Example 4

Pegylation of the Prodomain Allows for Less Aggregation After Purification from Inclusion Bodies but N Terminal Pegylation Interferes with Potency

SEQ ID NO: 4, 14,15 and 17 after elution from Ni-NTA beads as described above, were diluted into 20 mM Tris pH8 and left overnight at 4° C. The material was then concentrated, and either buffer exchange was performed as described above or the material was passed over a Superdex® 75 or 200 brand sizing column equilibrated in 20 mM Tris, pH 8, 40 mM NaCl or 20 mM phosphate buffer, pH8. Fractions were combined and concentrated. An IC50 value was estimated or determined by incubation of ADAM8 with varying concentrations of prodomain.

A version of SEQ ID NO: 14, 15 or 17, having a cysteine near its N or C terminus was solubilized from inclusion bodies and purified with Ni-NTA beads as described in Example 1 and then passed through a Zeba™ brand spin column equilibrated in urea and phosphate buffer. An excess 5 kDa maleimide PEG (Jenkem Technology, USA, Dallas, Tex.) was added. After 24 hours, the reaction progress was terminated by addition of TCEP to 10-20 mM. The TCEP was incubated with the prodomain for 24 more hours, after which time, material was either passed through an ion exchange column to remove the excess unreacted peg material or was directly refolded and then passed over a Superdex® G75 brand column equilibrated in 20 mM phosphate, pH 8. Fractions containing the pure pegylated material were concentrated. Purified fractions were frozen at −60° C. after addition of glycerol. In Table 5 above, SEQ ID NO:4, SEQ ID NO:17 or SEQ ID NO:15, had non-linear dose response curves. Increasing the concentration of prodomain for SEQ ID NO:4, SEQ ID NO:15 and SEQ ID NO:17 did not increase the inhibition. With the pegylated SEQ ID NO:15, SEQ ID NO:14, and SEQ ID NO:17 and with SEQ ID NO:2 and SEQ ID NO:14, there was no aggregation as indicated by a proper dose response curve. Unexpectedly, while SEQ ID NO:15 which was pegylated at the C-terminus, had an IC50 value close to 2 uM, pegylation near the N-terminus, increased the IC50 to over 20 uM. This was not the case for the ADAM10 or ADAM9 prodomains, where IC50s of pegylated prodomains near the N or C terminus where very similar.

### 7.5. Example 5

Cysteine Modifications

WT prodomain (SEQ ID NO:14) was passed over a Zeba™ brand desalting spin column equilibrated in urea and phosphate buffer. Several mg of prodomain was reacted at 4° C. with an excess of 2 kDa maleimide PEG (JenKem, USA) that can specifically react with sulfhydryl groups. After 24 hours, TCEP was added to 10 mM and the reaction was allowed to sit for an additional 24 hour at 4° C. The material was then passed over an ion exchange column to remove the excess un-reacted peg and then refolded, concentrated, and passed over a Superdex® G75 brand column equilibrated in 20 mM phosphate, pH 8. Fractions containing the pure pegylated material were concentrated. Purified fractions were assayed as described above. The IC50 of the pegylated prodomain was similar to the prodomain without pegylation, indicating that chemical modification of the cysteines, with something even as large as 2 k Peg does not interfere with the inhibitory properties of the prodomain.

### 7.6. Example 6

Cleavage Experiments and Identification of a Novel Furin Site

There is a known upstream site in the prodomain of ADAM8 and that it is cleaved by furin. However, Hall et al, 2009, demonstrated that the prodomain beginning at A45, which is cleaved at this upstream site, still retains the ability to bind to the catalytic domain of ADAM8. We made mutants at the upstream site at either aa44 or aa 41 and 44. To do the cleavage assays, 20-40 μg of each prodomain was reacted in a volume of approximately 100 μl in 1.7 ml Eppendorf tubes with furin. The buffer was 20 mM Tris pH 8 with 10 mM CaCl₂. Samples were incubated at 37° C. and a time course was run by removing approximately 20-30 μl of solution and quenching with 10 μl of a 4× solution of loading dye. Samples were run on a 16% Nowex gel and stained with SimplyBlue SafeStain (Thermo Fisher Scientific). These mutants were resistant to furin degradation or furin cleavage (FIG. 6 and FIG. 7).

During the course of these experiments, we found that some of the prodomains were being processed at more than one place. We found that the prodomain has an as yet unreported downstream furin site at amino acids 73-76. Interestingly, mutant SEQ ID NO:15 was resistant to furin cleavage at the downstream site, even though it was only mutated at the upstream site. However, this was not the case for the pegylated SEQ ID NO:15 as it was cleaved at the downstream site by furin. However, even though degradation did occur, the processing was much slower for the pegylated SEQ ID NO:15 as the wild type prodomain, SEQ ID NO:14, was completely degraded in 30 min, whereas, even after 100 min, there was still a significant amount of non-processed pegylated SEQ ID NO:15. (FIG. 6)

### 7.7. Example 7

The Human Prodomains of ADAM8 Inhibited Cellular

Shedding Events In Vitro

PANC 1 cells (20,000) stably transfected with CD23 containing an alkaline phosphatase tag are plated in a 96 well plate with growth medium and serum and prodomains or vehicle control are added from 3 nM-50 μM. Media is removed and a substrate for alkaline phosphatase is added. Protein levels are quantified by scanning the plate for detection of colorimetric or fluorescent signals.

### 7.8. Example 8

ADAM8 prodomains are not processed by ADAM8

Pegylated SEQ ID NOs: 14 and 15 and non-pegylated SEQ ID NO:2, 50ug, were diluted into buffer four-fold containing 20 mM Tris, pH8 and 10 mM Calcium Chloride. Reaction mixtures were divided into two and then either a buffer control or ADAM8 was diluted 30 fold, and then added to each prodomain at a final concentration of 90 nM. The reactions were incubated at 37° C. for 1 hr after which samples were quenched with loading dye and analyzed by SDS PAGE as described above. In FIG. 8, is a gel showing that under these reaction conditions, there was no processing or degradation of the prodomains.

### 7.9. Example 9

Furin and Autocatalytic Cleavage Resistant Mutants Had Good Pharmacokinetic Properties

Polypeptides selected from pegylated SEQ ID NOs: 2, 4-9, 14-17, and non-pegylated SEQ ID NOs: 2, 4-9, 14-17 are injected i.p. into 2-3 Balb/C mice along with vehicle control (20 mM phosphate, pH8, and 10% glycerol). After 72 hours, blood is collected via cardiac puncture and sera is prepared. Prodomain levels are measured by first preparing standard curves where varying known amounts of each prodomain are spiked into sera and ADAM8 in the presence of PEPDAB013 in buffer containing 20 mM Tris, 10 mM CaCl₂ and Brij-35 (0.001%). The percent inhibition of ADAM8 is calculated and a plot of prodomain concentration vs. the % inhibition gives a standard curve. Sera Samples from mice injected with either a vehicle control (no inhibition) or compounds, are added to PEPDAB013 as described above and the % inhibition of ADAM8 is measured. Using the standard curve, the amount of active prodomain can be calculated.

### 7.10. Example 10

Acute Liver Injury and Fibrosis Model

18 male, 8-week-old BALB/c mice are randomized into study groups based on body weight. Treatment with CCl₄ solution was initiated in all mice and continued for a duration of 2 weeks. Therapeutic treatments coincide with CCl₄ initiation. Compound administration and CCl₄ solution are at least 4 hours apart) for this two-week prophylactic dosing study, as outlined in Table 6.

TABLE 6
Study Groups and Treatments

| Study | Mice | | |
|---|---|---|---|
| Group | per | | |
| Number | group | Fibrosis Induction | Test Article (TA) |
| 1 | 6 | 20% CCl₄, BIW for 0-2 | Vehicle, i.p., EOD for 7 doses, |
| | | weeks | 0-2 weeks |
| 2 | 6 | 20% CCl₄, BIW for 0-2 | SEQ ID NOs: 2, 4-9, |
| | | weeks | 14-17, i.p., EOD for |
| | | | 7 doses, 0-2 weeks |

*** BIW: biweekly;
EOD: every other day

At the end of the study, approximately 400 μl whole blood is collected into serum separator tubes for ALT, AST, ALP, and bilirubin analyses. Following the terminal blood collection_each mouse has livers isolated, weighed, and cut into 4 sections as follows: a central section of the left lobe was formalin fixed (FFPE) for histology analysis; and three (3) smaller, separate sections, one from the remaining left lobe and two from the right lobe (˜50-100 mg each) are snap frozen and stored at −0° C.

Histology Analysis: Liver fibrosis scoring of the H&E/PSR stained liver sections from FFPE livers harvested at termination is provided by Board Certified Veterinary Pathologist (DVM). Scoring includes fibrosis and other pertinent observations such as necrosis and inflammation.

### 7.11. Example 11

In Vivo Studies: Tumor Xenograft Model

To determine whether the ADAM8 prodomain has therapeutic potential for tumor growth inhibition in vivo, efficacy studies are performed using derived xenograft tumor models in athymic mice. Briefly, cells, 1×10⁷ cells, are implanted s.c. into the flanks of 6-week-old female athymic mice. Tumor sizes in two dimensions are measured with calipers, and volumes are calculated. The treatments start when the tumor size has reached approximately 200 mm³. Vehicle control and ADAM8 prodomain peptide alone at two doses are given as determined from pharmacokinetic data. Each treatment group (n=6-12 per group) is monitored for up to 7-8 weeks. Body weights and tumors are measured twice weekly, and tumor growth and regression rates are determined. Animals are euthanatized at the end of the experiment and the liver, heart, visceral fat, kidneys, and brain are retained and examined for tissue damage and toxicity.

The ADAM8 prodomain peptide is used in a suitable dose in combination with other cancer agents in the xenograft tumor model. Cancer agents, alone or in combination with ADAM8 prodomain peptides, are administered and the experiment is performed as described above.

### 7.12. Example 12

Inhibition of Asthma with Prodomain Peptides

Asthma model. To determine whether the ADAM10 modulating peptides of the presently disclosed subject matter have therapeutic potential in asthma, efficacy studies are performed using an ovalbumin model that is TH₂ dependent. Briefly, a mast cell/IgE dependent mouse model is used. This model was used previously to test other less selective small molecule ADAM10 inhibitors (Mathews et al., 2011). 10 μg ovalbumin is injected i.p. into 6 female Balb/C mice per group on days 1, 3, 5, 7, 9, 11, and 13. Pegylated ADAM10 modulating peptides (2.5 mg/kg) or a vehicle control is given intranasally or i.p. every other day or every 3 days. An ovalbumin intranasal challenge (20 μg) is given on days 40, 43, and 46. On day 47, airway resistance is measured after the final challenge. Mice are anesthetized by i.p. injection of 207 mg/kg of ketamine and 42 mg/kg of xylazine. After cannulization and paralyzation, mice are ventilated and baseline lung function is measured. Mice are then exposed to acetyl beta methylcholine chloride. Newtonian resistance, tissue damping, and tissue elastance are measured. Following determination of lung function, bronchial aveloral lavage fluid is collected and saved for cytokine and chemokine analysis. Cells are pelleted, stained, and counted to determine types and amounts present. All statistics are done using the two-tailed T test, with Bonferroni correction when multiple comparisons are made. Additionally, the maximum tolerated dose is determined through acute tolerability studies in mice. Mice are monitored daily for clinical signs of distress in response to different drug doses Animals are euthanatized at the end of the experiment and the liver, intestines, heart, visceral fat, kidneys, and brain are retained and examined for tissue damage and toxicity.

### 7.13. Example 13

Inhibition of Allergies with Prodomain Peptides

House dust mite antigen (HDM) extract (Greer Laboratories, Lenoir, NC) is re-suspended in normal saline and introduced by intranasal instillation, with the mice lightly anesthetized with 5% isoflurane, using a 20-200 μL pipette tip. The acute HDM protocol used 100 μg HDM (D. pteronyssinus or D. farinae) in 50 μL saline on days 0-4 and day 11. Saline-exposed control mice receive 50 μL sterile saline. The 4-week models use 10 μg or 25 μg D. pteronyssinus in 35 μL of saline. Mice are sensitized for 5 consecutive days weekly (from Monday to Fridays) for 4 weeks. The mice in the 8-week model are sensitized to 25 μg D. pteronyssinus for 5 consecutive days on week 1, followed by every other weekday (Monday, Wednesday and Friday) from weeks 2 to 8. Saline-exposed control mice in the 2-week, 4-week and 8-week models are handled in a similar fashion but are only treated with sterile saline. Evaluation of the endpoint metrics occur 24 hours after the last HDM or saline exposure.

### 7.14. Example 14

Inhibition of Wound Healing with Prodomain Peptides

C57/B16 (8-10 weeks old; 8 per group; 2 groups) mice are anesthetized with a single i.p. injection of ketamine/xylazine. The hair on the back is shaved and the skin wiped with 70% ethanol. Two full-thickness excisional wounds (4 mm diameter) are created on the back of each animal by excising the skin and panniculus carnosus as previously described. The wounds are allowed to dry to form a scab Animals are administered via IP injection or with a topical application of vehicle control (20 mM Tris pH 8, 10% glycerol) or prodomain every 1-3 days for two weeks. Mice are sacrificed at different time points after wounding and the complete wounds, including the epithelial margins, are isolated. Wounds are bisected in caudocranial direction and the tissue embedded in O.C.T. Compound (Tissue Tek, Vogel, Giessen, Germany), and used for immunohistochemistry. Histological analyses are performed on serial sections from the central portion of the wound. Cryosections (5 μm) of the wounds are stained with hematoxylin and eosin (H&E; Shandon, Frankfurt, Germany), documented, and measured using a Leica microscope (DMLB, Wetzlar, Germany)

### 8. REFERENCES

All references listed below, as well as all references cited in the instant disclosure, including but not limited to all patents, patent applications and publications thereof, scientific journal articles, and database entries (e.g., GENBANK® biosequence database entries and all annotations available therein) are incorporated herein by reference in their entireties to the extent that they supplement, explain, provide a background for, or teach methodology, techniques, and/or compositions employed herein.
Banerjee et al. (2011) American Journal of Physiology-Gastrointestinal and Liver Physiology 300:G273-282.
Bergin et al. (2008) Journal of Biological Chemistry 283:31736-31744.
Deuss et al. (2008) Current Alzheimer Research 5:187-201.
Edwards et al. (2008) Molecular Aspects of Medicine 29(5):258-289.
Guaiquil et al. (2009) Molecular and Cellular Biology 29(10):2694-2703.
Hall et al. (2009) Biosci Rep. 29 (4): 217-228.
Jefferson et al. (2013) Cellular and Molecular Life Sciences 70:309-333.
Kyte & Doolittle (1982) Journal of Molecular Biology 157:105-132.
Ludwig et al. (2005) Combinatorial Chemistry & High Throughput Screening 8(2):161-171.
Maretzky et al. (2017) The Biochemical Journal 474(9):1467-1479.
Mauch et a. (2014) The Journal of Investigative Dermatology 130:2120-2130.
Miller et al. (2011) Integr Biol (Camb) 3:422-438.
Moss et al. (2008) Nature Clinical Practice Rheumatology 4(6):300-309.
Moss et al. (2011) Journal of Biological Chemistry 286(47):40443-40451.
Pruessmeyer & Ludwig (2009) Seminars in Cell & Developmental Biology 20(2):164-174.
Roychaudhuri et al. (2014) Journal of Immunology 193:2469-2482.
Sahin et al. (2004) The Journal of Cell Biology 164(5):769-779.
Schutte et al. (2014) Proceedings of the National Academy of Sciences of the United States of America 111:12396-12401.
Srinivasan et al. (2014) Journal of Biological Chemistry 289(48):33673-33688.
Vazeille et al. (2011) Role of meprins to protect ileal mucosa of Crohn's disease patients from colonization by adherent-invasive E. coli, PLoS One 6:e21199.
Wang et al. (2018) A Disintegrin and A Metalloproteinase-9 (ADAM9): A Novel Proteinase Culprit with Multifarious Contributions to COPD. American Journal of Respiratory and Critical Care Medicine. Jun 4. doi: 10.1164/rccm.201711-2300OC. [Epub ahead of print]
Wong et al. (2015) Journal of Biological Chemistry 290(19):12135-12146.
Wong et al. (2016) Scientific Reports 6:35598.
Zhou et al. (2006) Cancer Cell 10(1):39-50.

It will be understood that various details of the presently disclosed subject matter can be changed without departing from the scope of the presently disclosed subject matter. Furthermore, the foregoing description is for the purpose of illustration only, and not for the purpose of limitation.

### 9. NUMBERED EMBODIMENTS

The following numbered embodiments are includings within the scope of the disclosure.

1. A peptide comprising the amino acid sequence set forth in SEQ ID NOS: 3, 18, or 19, wherein relative to the amino acid sequence set forth in SEQ ID NO: 13, the peptide includes one or more amino acid substitutions and/or modifications at an amino acid position selected from the group consisting of amino acids 41, 43, 44, 73, 74, 77, 67, 91, 105, 124, 152-164, and 167, such that the peptide is auto-catalytically cleaved less, is less sensitive to furin cleavage, is less susceptible to oxidation and/or disulfide bond formation, less susceptible to degradation by ADAM8, or any combination thereof, as compared to a peptide without the one or more amino acid substitutions and/or modifications.

2. The peptide of claim 1, wherein the peptide comprises an amino acid sequence that is at least 90% identical to SEQ ID NO: 2, and further wherein as compared to SEQ ID NO: 13, the amino acid sequence comprises one or more amino acid substitutions and/or modifications selected from the group consisting of:

(a) a substitution of arginine 41 to another amino acid, optionally alanine, serine, glycine, or lysine;

(b) a substitution of arginine 43 to another amino acid, optionally alanine, serine, glycine, or lysine;

(c) a substitution of arginine 44 to another amino acid, optionally alanine, serine, glycine, or lysine;

(d) a substitution of arginine 73 to another amino acid, optionally alanine, serine, glycine, or lysine;

(e) a substitution of lysine 74 to another amino acid, optionally alanine, serine, or glycine;

(f) a substitution of arginine 76 to another amino acid, optionally alanine, serine, glycine, or lysine;

(g) a substitution of glutamine 156 to another amino acid, optionally serine, alanine, glycine or asparagine;

(h) a substitution of glutamate 158 to another amino acid, optionally serine, alanine, glycine or asparagine;

(i) a substitution of glutamine 162 to another amino acid, optionally serine, alanine, glycine or asparagine;

(j) a substitution of amino acids 152, 153, 154, 155, 157, 159, 150, 161 to another amino acid;

(k) a substitution of cysteine 105 to another amino acid, optionally serine, alanine, or glycine;

(l) a substitution of cysteine 124 to another amino acid, optionally serine, alanine, or glycine;

(m)a substitution of cysteine 167 to another amino acid, optionally serine, alanine, or glycine; and

(n) a chemical modification of one, two, or all three of cysteines 105, 124, and 167, or any combination thereof.

3. The peptide of claim 1, wherein as compared to SEQ ID NO: 13, the amino acid sequence has:

(i) a substitution at one, two, three, four, or all five of amino acids 41, 43, 44, 73, and 76, wherein each substitution is independently any amino acid other than the amino acid present in the corresponding position of SEQ ID NO: 2 at the corresponding position, optionally wherein each substitution is independently selected from the group consisting of alanine, serine, glycine, and lysine; or

(ii) a substitution at one or more of amino acids 156, 158 and 162, wherein each substitution is independently any amino acid other than the amino acid present in the corresponding position of SEQ ID NO: 2 at the corresponding position, optionally wherein each substitution is independently selected from the group consisting of asparagine, alanine, serine, and glycine; or

(iii)a substitution at one or more amino acids 152, 153, 154, 155, 157, 159, 150, 161, wherein each substitution is independently any amino acid other than the amino acid present in the corresponding position of SEQ ID NO: 2 at the corresponding position, optionally wherein each substitution is independently selected from the group consisting of any of the natural amino acids; or

(iv) a substitution at one, two, or all three of amino acids 105, 124, and 167, wherein each substitution is independently any amino acid other than cysteine, optionally wherein each substitution is independently selected from the group consisting of serine, alanine, and glycine;

(v) or any combination thereof.

4. The peptide of SEQ ID NO: 3, where X 35 of SEQ ID NO: 13 is arginine; arginine 41 is replaced with lysine; arginine 44 of SEQ ID NO: 13 is replaced with alanine, glycine, or lysine; and/or cysteines 105, 124, and 167 of SEQ ID NO: 13 are replaced with serines, or any combination thereof.

5. The peptide of any one of claims 1-4, wherein one, two, or all three of cysteines 105, 124, and 167 of SEQ ID NO: 13 is chemically modified at its a sulfhydryl group, optionally wherein the sulfhydryl group(s) is/are chemically modified by addition of a maleimide ester, an α-halocarbonyl, a thiosulfonate, a disulfide derivative, or any combination thereof.

6. The peptide of any one of claims 1-4, wherein with reference to SEQ ID NO: 13, the amino acid sequence comprises a substitution of one or more of arginine 41, 43, 44, 73, and/or 76 to an amino acid other than arginine, a substitution of lysine 74 to an amino acid other than lysine, a substitution of one or more of cysteines 105, 124, and 167 to an amino acid other than cysteine, optionally serine, or any combination thereof.

7. The peptide of any one of claims 1-4, further comprising a pegylated cysteine added within 20 amino acids of the N-terminus, within 20 amino acids of the C-terminus, or both, optionally wherein one or both of the pegylated cysteines comprise a PEG group having a molecular weight of about 1 kiloDalton (kDa) to about 40 kDa.

8. The peptide of any one of claims 1-4, wherein with reference to SEQ ID NO: 13, the amino acid sequence comprises a substitution of at least one of arginine 41, arginine 43, arginine 44, arginine 73, or arginine 76 to an amino acid other than arginine, and substitutions of cysteines 105, 124, and 167 to serine.

9. The peptide of any one of claims 1-4, wherein with reference to SEQ ID NO: 13, the amino acid at position 105, 124, and/or 167 is cysteine and further wherein cysteine 105, 124, and/or 167 is pegylated, optionally wherein the pegylation comprises a PEG group having a molecular weight of about 1 kDa to about 40 kDa.

10. The peptide of any one of claims 1-4, wherein with reference to SEQ ID NO: 13, one or more of cysteines 105, 124, and 167 comprises a chemical modification with a maleimide ester.

11. The peptide of claim 10, further comprising modifying arginine 41, arginine 43, arginine 44, arginine 73, and/or arginine 76 of SEQ ID NO: 13 to increase resistance of the peptide to furin cleavage.

12. The peptide of any one of claims 1-4, wherein with reference to SEQ ID NO: 13, one or more of cysteines 105, 124, and 167 comprises a chemical modification resulting from reacting the one or more cysteines with a disulfide.

13. The peptide of claim 12, further comprising modifying arginine 41, arginine 43, arginine 44, arginine 73, and/or arginine 76 of SEQ ID NO: 13 to increase resistance of the peptide to furin cleavage.

14. The peptide of any one of claims 1-4, wherein the peptide comprises an amino acid sequence having a percent identity of at least 87% to any one of SEQ ID NOs: 2-9 or 14-20, optionally wherein the percent identity is at least 95%.

15. The peptide of claim 14, wherein the peptide comprises an amino acid sequence having 100% percent identity to any one of SEQ ID NOs: 2-9 or 14-20 over its full length.

16. The peptide of any one of claims 1-4, wherein the peptide further comprises one or more additional modifications selected from the group consisting of conservative amino acid substitutions, non-natural amino acid substitutions, D- or D,L-racemic mixture isomer form amino acid substitutions, amino acid chemical substitutions, carboxy- and/or amino-terminus modifications, glycosylations, carbohydrate additions, and conjugations to biocompatible molecules such as but not limited to fatty acids and peptides of interest.

17. The peptide of any one of claims 1-4, wherein with reference to SEQ ID NO: 13, the peptide further comprises a modification of at least one of cysteines 105, 124, and/or 167, wherein the modification comprises attachment of a maleimide ester or other chemical entity used to selective attach to a sulfhydryl group comprising at least one moiety selected from the group consisting of a PEG group, a fluorescent moiety, an alkyl moiety, a colorimetric moiety, a bifunctional moiety, a radiometric moiety, a carbohydrate moiety, a fatty acid moiety, a toxin, a therapeutic agent, a C-terminus cysteine, an N-terminus cysteine, optionally a chemotherapeutic agent, a linker, a peptide, or any combination thereof.

18. A composition comprising the peptide of any one of claims 1-17, wherein the composition is formulated for administration to a subject or is a pharmaceutical composition formulated for administration to a human

19. A fusion protein comprising the peptide of any one of claims 1-17.

20. The fusion protein of claim 19, wherein the peptide is conjugated to an agent selected from the group consisting of a therapeutic moeity, a diagnostic moiety, a detectable moiety, or any combination thereof, optionally wherein the peptide is conjugated to the agent via a linker molecule or via a peptide linkage.

21. The fusion protein of claim 20, wherein the therapeutic molecule is selected from the group consisting of a therapeutic antibody, a nanobody, an Fc fragment, a receptor, a toxin, a chemotherapeutic molecule, or any combination thereof.

22. A polypeptide comprising an amino acid sequence as set forth in any of SEQ ID NOs 3-9 or 14-20.

23. The polypeptide of claim 22, further comprising a tag, optionally a His tag, that can be employed for purification and/or isolation of the polypeptide from an expression system.

24. The polypeptide of claim 22, further comprising a recognition site for a protease between the tag and an amino acid of the polypeptide that can be employed for releasing the tag from the polypeptide by proteolytic cleavage.

25. A polypeptide comprising an amino acid sequence as set forth in any one of SEQ ID NOs 3-9 or 14-20 and further comprising an addition of one or more amino acids to the N-terminus of the polypeptide, an addition of one or more amino acids to the C-terminus of the polypeptide, or an addition of one or more amino acids to both the N-terminus and the C-terminus of the polypeptide, wherein the one or more amino acids comprises at least one cysteine residue that provides functionality to conjugate a moiety of interest to the polypeptide. In some embodiments the N-terminus or C-terminus cysteine is added at the end of the polypeptide. In other embodiments, it is inserted within 5 amino acids, 10 amino acids, or 20 amino acids from the N-terminus or C-terminus of the polypeptide.

26. The polypeptide of claim 25, wherein the one or more amino acids added to the N-terminus of the polypeptide comprise SEQ ID NO: 10 and/or SEQ ID NO: 11 and or the one or more amino acids added to the C-terminus of the polypeptide comprise SEQ ID NO: 10 and/or SEQ ID NO: 11.

27. The polypeptide of claim 25 or claim 26, further comprising a PEG group conjugated to a cysteine present in the one or more amino acids added to the N-terminus and/or the C-terminus of the polypeptide, wherein the PEG group enhances the proper folding of the polypeptide; stabilizes the polypeptide to auto-catalytic and/or furin cleavage; and/or prevents aggregation; relative to the polypeptide lacking a PEG group.

28. An isolated nucleic acid encoding the polypeptide of claims 1-4.

29. The isolated nucleic acid of claim 28, wherein the isolated nucleic acid comprises a nucleic acid sequence that is at least 97% identical to the nucleic acid sequence as set forth in SEQ ID NOs: 21-26.

30. An isolated nucleic acid encoding a polypeptide of claims 1-4, wherein the isolated nucleic acid comprises a nucleic acid sequence as set forth in SEQ ID NOs: 21-26.

31. A vector comprising the isolated nucleic acid of claim 28.

32. A recombinant host cell comprising the vector of claim 31.

33. The recombinant host cell of claim 32, wherein the host cell further comprises a gene encoding a chimeric antigen receptor (CAR).

34. A method for modulating an ADAM8 biological activity in a eukaryotic cell comprising an ADAM8 protein, the method comprising contacting the cell with the peptide of any one of claims 1-17 or the composition of claim 18 in an amount sufficient to inhibit the activity of the ADAM8 protein.

35. A method for modulating an ADAM8 biological activity in a eukaryotic cell comprising an ADAM8 protein, the method comprising contacting the cell with an isolated nucleic acid comprising a nucleic acid encoding the polypeptide of claims 1-4 or the vector of claim 32 under suitable conditions that the polypeptide is expressed in an amount sufficient to inhibit the activity of the ADAM8 protein.

36. The eukaryotic cell of claim 35, wherein the eukaryotic cell further comprises a gene encoding a chimeric antigen receptor (CAR).

37. A method for modulating an ADAM8 biological activity in a eukaryotic cell which comprises contacting the cell with a genome modifying enzyme, whereby the genome modifying enzyme modifies a nucleic acid encoding an amino acid sequence set forth in SEQ ID NO: 13, such that the nucleic acid expresses a peptide that includes one or more amino acid substitutions and/or modifications at an amino acid position selected from the group consisting of amino acids 41, 43, 44, 67, 73, 74, 77, 91, 105, 124, 152-164, and 167, such that the expressed peptide is auto-catalytically cleaved less, is less sensitive to furin cleavage, is less susceptible to oxidation and/or disulfide bond formation, or any combination thereof, as compared to a peptide without the one or more amino acid substitutions and/or modifications.

38. The eukaryotic cell of claim 37, wherein the eukaryotic cell further comprises a gene encoding a chimeric antigen receptor (CAR).

39. The method of any of claim 37 or 38, wherein the genome modifying enzyme is a transcription activator-like effector nuclease (TALEN); a zinc finger (ZF) protein; or a CRISPR associated protein and a guide nucleic acid.

40. A method for inhibiting an ADAM8 biological activity in a subject, the method comprising administering to the subject the peptide of any one of claims 1-17 or the composition of claim 18 in an amount and via a route sufficient to contact an ADAM8 polypeptide present in the subject, whereby an ADAM8 biological activity in a subject is modulated.

41. A method for inhibiting an ADAM8 biological activity in vivo, the method comprising administering to a subject a polypeptide comprising an amino acid sequence as set forth in any one of SEQ ID NOs 2-9 or 14-20 in an amount and via a route sufficient to inhibit an ADAM8 biological activity in vivo, optionally wherein the polypeptide is pegylated.

42. A method for inhibiting an ADAM8 biological activity associated with a disease or disorder in a subject, the method comprising contacting an ADAM8 protein present in the subject with an effective amount of the peptide of any one of claims 1-17 or the composition of claim 18, wherein the disease or disorder is selected from the group consisting of cancer, inflammation, asthma, allergies, lung injuries, diseases related to excessive osteoclast formation or activity, vascular diseases, fibrosis, Alzheimer's disease, a wound, and undesirable angiogenesis, or wherein the subject has a predisposition thereto.

43. The method of claim 42, wherein the disease or disorder comprises a liver injury, optionally a liver injury associated with liver fibrosis, and the effective amount is sufficient to reduce a biological activity of an MMP9 or ADAM gene product in the subject's liver.

44. The method of claim 43, wherein the disease or disorder results at least in part from excess cell proliferation associated with an ADAM8 biological activity.

45. The method of any one of claims 40-44, wherein the subject has a disease or disorder characterized at least in part by undesirably high ADAM8 biological activity or ADAM8 protein expression.

46. A method for decreasing inflammation, the method comprising administering to a subject an effective amount of the peptide of any one of claims 1-17 or the composition of claim 18.

47. A method for inhibiting cell invasion and/or metastasis associated with undesirable ADAM8 biological activity in a subject, the method comprising administering to the subject an effective amount of the peptide of any one of claims 1-17 or the composition of claim 18.

48. A method for inhibiting the release of a substrate of ADAM8 in vivo, the method comprising administering to a subject in need thereof a peptide comprising an amino acid sequence as set forth in any one of SEQ ID NOs 2-9 or 14-20, optionally wherein the peptide is pegylated.

49. The method of any one of claims 40-48, wherein the peptide of any one of claims 1-17 or the composition of claim 18 is formulated for administration via a route selected from the group consisting of inhalation, oral administration, intraadiposal administration, intraarterial administration, intraarticular administration, intracranial administration, intradermal administration, intralesional administration, intramuscular administration, intranasal administration, intraocular administration, intrapericardial administration, intraperitoneal administration, intrapleural administration, intraprostatic administration, intrarectal administration, intrathecal administration, intratracheal administration, intratumoral administration, intraumbilical administration, intravaginal administration, intravenous administration, intravesicular administration, intravitreal administration, subconjunctival administration, subcutaneous administration, sublingual administration, topical administration, transbuccal administration, oropharyngeal aspiration, and transdermal administration.

50. The method of claim 49, wherein the peptide of any one of claims 1-17 or the composition of claim 18 is delivered in a lipid composition, optionally a liposome or an ethosome; in a creme; via a catheter; via lavage; via infusion, optionally via continuous infusion; via inhalation; via injection; via local delivery; via localized perfusion; by bathing target cells directly; or any combination thereof.

51. A method for inhibiting ADAM8 biological activity associated with a disease or disorder in a subject, the method comprising contacting the subject with an isolated nucleic acid encoding the polypeptide of claims 1-4 or the vector of claim 31 under suitable conditions that the polypeptide is expressed in an amount sufficient to inhibit the activity of the ADAM8 protein, wherein the disease or disorder is selected from the group consisting of cancer, inflammation, asthma, allergies, lung injuries, diseases related to excessive osteoclast formation or activity, vascular diseases, fibrosis, Alzheimer's disease, a wound, and undesirable angiogenesis, or wherein the subject has a predisposition thereto.

52. The method of claim 51, wherein the isolated nucleic acid is delivered to the cell in a cationic polymer, a cell penetrating polypeptide, a dendrimer, or a liposome.

53. The method of claim 51, wherein the vector is an adenovirus vector (AV), adeno-associated vector (AAV), a herpes simplex vector (HSV), or a retrovirus vector.

54. A method for inhibiting ADAM8 biological activity associated with a disease or disorder in a subject, the method comprising contacting the subject with a genome modifying enzyme, whereby the genome modifying enzyme modifies a nucleic acid encoding an amino acid sequence set forth in SEQ ID NO: 13, such that the nucleic acid expresses a peptide that includes one or more amino acid substitutions and/or modifications at an amino acid position selected from the group consisting of amino acids 41, 43, 44, 67, 73, 74, 77, 91, 105, 124, 152-164, and 167, such that the expressed peptide is auto-catalytically cleaved less, is less sensitive to furin cleavage, is less susceptible to oxidation and/or disulfide bond formation, or any combination thereof, as compared to a peptide without the one or more amino acid substitutions and/or modifications and the polypeptide is expressed in an amount sufficient to inhibit the activity of the ADAM8 protein, wherein the disease or disorder is selected from the group consisting of cancer, inflammation, asthma, allergies, lung injuries, diseases related to excessive osteoclast formation or activity, vascular diseases, fibrosis, Alzheimer's disease, a wound, and undesirable angiogenesis, or wherein the subject has a predisposition thereto.

55. The method of claim 54, wherein the genome modifying enzyme is a transcription activator-like effector nuclease (TALEN); a zinc finger (ZF) protein; or a CRISPR associated protein and a guide nucleic acid.

56. A method for attaching a peptide comprising an amino acid sequence as set forth in any of SEQ ID NOs: 2-9 or 14-20 to an antibody, antibody fragment, or other protein, the method comprising conjugating the peptide to the antibody, antibody fragment, or other protein, optionally via a linker, further optionally via a peptide linker.

57. A polypeptide comprising an amino acid sequence as set forth in any one of SEQ ID NOs 2-9 or 14-20, wherein a cysteine that corresponds to an amino acid residue of SEQ ID NO: 13 selected from the group consisting of cysteines 105, 124, and 167, or any combination thereof, is conjugated to one or more moieties that improve the inhibitory potency, solubility, and/or a pharmacokinetic property of the polypeptide relative to the polypeptide lacking the moiety, and/or is conjugated to one or more chromophores, fluorophores, and/or radionucleotides.

58. A polypeptide comprising an amino acid sequence as set forth in one of SEQ ID NOs: 2-9 or 14-20, for use in treating a subject with a disorder associated with undesirable ADAM8 biological activity, optionally wherein the disorder associated with undesirable ADAM8 biological activity is selected from the group consisting of cancer, inflammation, asthma, allergies, lung injuries, diseases related to excessive osteoclast formation or activity, vascular disease, fibrosis, Alzheimer's disease, a wound, and undesirable angiogenesis, optionally wherein the polypeptide is pegylated at one or more of the amino acids of SEQ ID NOs: 2-9 or 14-20.

59. The polypeptide of claim 58, wherein the polypeptide is pegylated.

60. The polypeptide of any one of claim 58 or 59, wherein the subject is a human.

61. A polypeptide comprising an amino acid sequence as set forth in one of SEQ ID NOs: 2-9 or 14-20 for use in preventing development of and/or reducing the severity of at least one symptom of a disorder associated with undesirable ADAM8 biological activity, optionally wherein the disorder associated with undesirable ADAM8 biological activity is selected from the group consisting of cancer, inflammation, asthma, allergies, vascular disease, fibrosis, Alzheimer's disease, a wound, and undesirable angiogenesis, in a subject in need thereof, optionally wherein the subject has a predisposition to development the at least one symptom.

62. The polypeptide of claim 61, wherein the polypeptide is pegylated.

63. The polypeptide of any of claim 61 or claim 62, wherein the subject is a human

64. The polypeptide of SEQ ID NO: 2-9 or 14-20 which is used in combination with existing or emerging drugs for use in preventing development of and/or reducing the severity of at least one symptom of a disorder associated with undesirable ADAM8 biological activity, optionally wherein the disorder associated with undesirable ADAM8 biological activity is selected from the group consisting of cancer, inflammation, asthma, allergies, lung injuries, diseases related to excessive osteoclast formation or activity, vascular disease, fibrosis, Alzheimer' s disease, a wound, and undesirable angiogenesis, in a subject in need thereof.

65. A polypeptide comprising an amino acid sequence as set forth in one of SEQ ID NO: 2-9 or 14-20 for use in preventing development of and/or reducing the severity of at least one symptom of a disorder associated with undesirable ADAM8 biological activity, optionally wherein the disorder associated with undesirable ADAM8 biological activity is selected from the group consisting of cancer, inflammation, asthma, allergies, vascular disease, fibrosis, Alzheimer's disease, a wound, and undesirable angiogenesis, in a subject in need thereof, optionally wherein the subject has a predisposition to development the at least one symptom.

66. The polypeptide of claim 65, wherein the polypeptide is pegylated.

67. The polypeptide of any one of claim 65 or claim 66, wherein the subject is a human

It should be understood that the above description is only representative of illustrative embodiments and examples. For the convenience of the reader, the above description has focused on a limited number of representative examples of all possible embodiments, examples that teach the principles of the disclosure. The description has not attempted to exhaustively enumerate all possible variations or even combinations of those variations described. That alternate embodiments may not have been presented for a specific portion of the disclosure, or that further undescribed alternate embodiments may be available for a portion, is not to be considered a disclaimer of those alternate embodiments. One of ordinary skill will appreciate that many of those undescribed embodiments, involve differences in technology and materials rather than differences in the application of the principles of the disclosure. Accordingly, the disclosure is not intended to be limited to less than the scope set forth in the following claims and equivalents.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world. It is to be understood that, while the disclosure has been described in conjunction with the detailed description, thereof, the foregoing description is intended to illustrate and not limit the scope. Other aspects, advantages, and modifications are within the scope of the claims set forth below. All publications, patents, and patent applications cited in this specification are herein incorporated by reference as if each individual publication or patent application were specifically and individually indicated to be incorporated by reference.

```
SUMMARY OF SEQUENCES
(GENBANK® Accession No. NP_001100.3; human ADAM8)
SEQ ID NO: 1
MRGLGLWLLGAMMLPAIAPSRPWALMEQYEVVLP [symbol] RLPGPRVRRALPSHLGLHPERVS
YVLGATGHNFTLHLRKNRDLLGSGYTETYTAANGSEVTEQPRGQDHCFYQGHVEGYPD
SAASLSTCAGLRGFFQVGSDLHLIEPLDEGG [symbol] GGRHAVYQAEHLLQTAGTCGVSDDSLG
SLLGPRTAAVFRPRPGDSLPSRETRYVELYVVVDNAEFQMLGSEAAVRHRVLEVVNHVD
KLYQKLNFRVVLVGLEIWNSQDRFHVSPDPSVTLENLLTWQARQRTRRHLHDNVQLITG
VDFTGTTVGFARVSAMCSHSSGAVNQDHSKNPVGVACTMAHEMGHNLGMDHDENVQ
GCRCQERFEAGRCIMAGSIGSSFPRMFSDCSQAYLESFLERPQSVCLANAPDLSHLVGGPV
CGNLFVERGEQCDCGPPEDCRNRCCNSTTCQLAEGAQCAHGTCCQECKVKPAGELCRPK
KDMCDLEEFCDGRHPECPEDAFQENGTPCSGGYCYNGACPTLAQQCQAFWGPGGQAAE
ESCFSYDILPGCKASRYRADMCGVLQCKGGQQPLGRAICIVDVCHALTTEDGTAYEPVPE
GTRCGPEKVCWKGRCQDLHVYRSSNCSAQCHNHGVCNHKQECHCHAGWAPPHCAKLL
TEVHAASGSLPVFVVVVLVLLAVVLVTLAGIIVYRKARSRILSRNVAPKTTMGRSNPLFH
QAASRVPAKGGAPAPSRGPQELVPTTHPGQPARHPASSVALKRPPPAPPVTVSSPPFPVPV
YTRQAPKQVIKPTFAPPVPPVKPGAGAANPGPAEGAVGPKVALKPPIQRKQGAGAPTAP
(amino acids 17-196 of GENBANK® Accession No. NP_001100.3
with an N-terminal Met) (SEQ 39)
SEQ ID NO: 2
MIAPSRPWALMEQYEVVLP [symbol] RLPGPRVRRALPSHLGLHPERVSYVLGATGHNFTLHLRK
NRDLLGSGYTETYTAANGSEVTEQPRGQDHCFYQGHVEGYPDSAASLSTCAGLRGFFQV
GSDLHLIEPLDEGG [symbol] GGRHAVYQAEHLLQTAGTCGVSDDSLGSLLGPRTAAVFRPRPGDS
LPS
(consensus sequence)
SEQ ID NO: 3
MIAPSRPWALMEQYEVVLP [symbol] RLPGP [symbol] V [symbol] ALPSHLGLHPERVSYVLGATGHNFTLHLRKN
RDLLGSGYTETYTAANGSEVTEQPRGQDH [symbol] FYQGHVEGYPDSAASLST [symbol] AGLRGFFQVG
SDLHLIEPLDEGG [symbol] GGR [symbol] GT [symbol] GVSDDSLGSLLGPRTAAVFRPRPGDSLPS
(embodiment of SEQ ID NO: 3)
SEQ ID NO: 4
MIAPSRPWALMEQYEVVLP [symbol] RLPGPRV [symbol] ALPSHLGLHPERVSYVLGATGHNFTLHLRK
NRDLLGSGYTETYTAANGSEVTEQPRGQDH [symbol] FYQGHVEGYPDSAASLST [symbol] AGLRGFFQV
GSDLHLIEPLDEGG [symbol] GGR [symbol] GT [symbol] GVSDDSLGSLLGPRTAAVFRPRPGD
SLPS
(embodiment of SEQ ID NO: 3)
SEQ ID NO: 5
MIAPSRPWALMEQYEVVLP [symbol] RLPGP [symbol] V [symbol]
LPSHLGLHPERVSYVLGATGHNFTLHLRKNRDLL
GSGYTETYTAANGSEVTEQPRGQDH [symbol] FYQGHVEGYPDSAASLST [symbol] AGLRGFFQVGSDLH
LIEPLDEGG [symbol] GGR [symbol] GT [symbol] GVSDDSLGSLLGPRTAAVFRPRPGDSLPS
(embodiment of SEQ ID NO: 3)
SEQ ID NO: 6
MIAPSRPWALMEQYEVVLP [symbol] RLPGP [symbol] V [symbol] ALPSHLGLHPERVSYVLGATGHNFTLHLR
KNRDLLGSGYTETYTAANGSEVTEQPRGQDH [symbol] FYQGHVEGYPDSAASLST [symbol] AGLRGFFQ
VGSDLHLIEPLDEGG [symbol] GGR [symbol] GT [symbol] GVSDDSLGSLLGPRTAAVFRPRPG
DSLPS
(embodiment of SEQ ID NO: 3)
SEQ ID NO: 7
MIAPSRPWALMEQYEVVLP [symbol] RLPGP [symbol] V [symbol] ALPSHLGLHPERVSYVLGATGHNFTLHLR
KNRDLLGSGYTETYTAANGSEVTEQPRGQDH [symbol] FYQGHVEGYPDSAASLST [symbol] AGLRGFFQ
VGSDLHLIEPLDEG [symbol] EGGR [symbol] GT [symbol] GVSDDSLGSLLGPRTAAVFRPRPG
DSLPS
(embodiment of SEQ ID NO: 3)
SEQ ID NO: 8
MIAPSRPWALMEQYEVVLP [symbol] RLPGP [symbol] V [symbol] ALPSHLGLHPERVSYVLGATGHNFTLHLR
KNRDLLGSGYTETYTAANGSEVTEQPRGQDH [symbol] FYQGHVEGYPDSAASL [symbol] TSAGLRGFFQ
VGSDLHLIEPLDEGG [symbol] GGR [symbol] GT [symbol] GVSDDSLGSLLGPRTAAVFRPRPG
DSLPS
(embodiment of SEQ ID NO: 3)
SEQ ID NO: 9
MIAPSRPWALMEQYEVVLP [symbol] RLPGP [symbol] V [symbol] ALPSHLGLHPERVSYVLGATGHNFTLHLRK
NRDLLGSGYTETYTAANGSEVTEQPRGQDH [symbol] FYQGHVEGYPDSAASLST [symbol] AGLRGFFQV
GSDLHLIEPLDEGG [symbol] GGR [symbol] GT [symbol] GVSDDSLGSLLGPRTAAVFRPRPGD
SLPS
SEQ ID NO: 10
GSCGS
SEQ ID NO: 11
GSGSC
UniProtKB - Q14C66 (Q14C66_HUMAN)
SEQ ID NO: 12
                10 20 30 40 50
MRGLGLWELG AMMLPAIAPS RPWALMEQYE VVLPRRLPGP RVRRALPSHL
                60 70 80 90 100
GLHPERVSYV LGATGHNFTL HLRKNRDLLG SGYTETYTAA NGSEVTEQPR
              110 120 130 140 150
GQDHGEYQGH VEGYPDSAAS LSTCAGLRGF FQVGSDLHLI EPLDEGGEGG
              160 170 180 190 200
RHAVYQAEHL LQTAGTCGVS DDSLCSLLGP RTAAVFRPRP GDSLPSRETR
              210 220 230 240 250
YVELYVVVDN AEFQMLGSEA AVRHRVLEVV NHVDKLYQKL NFRVVLVGLE
              260 270 280 290 300
IWNSQDRFHV SPDPSVTLEN LLTWQARQRT RRHLHDNVQL ITGVDFTGTT
              310 320 330 340 350
VGFARVSAMC SHSSGAVNQD HSKNPVGVAC TMAHEMGHNL GMPHDENVQG
              360 370 380 390 400
CRCQERFEAG RCIMAGSIGS SEPRMESDCS QAYLESELER PQSVCLANAP
              410 420 430 440 450
DLSHLVSGPV CGNLFVERGE QCDCGPPEDC RNRCCNSTTC QLAEGAQCAH
              460 470 480 490 500
GTCCQECKVK PAGELCRPKK DMCDLEEECD GRHPECPEDA FQENGTPCSG
              510 520 530 540 550
GYCYNGACPT LAQQCQAFWG PGGQAAEESC FSYDILPGCK ASRYRADMCG
              560 570 580 590 600
VLQCKGGQQP LGRAICIVDV CHALTTEDGT AYEPVPEGTR CGPEKVCWKG
              610 620 630 640 650
RCQDLHVYRS SNCSAQCHNH GVCNHKQECH CHAGWAPPHC AKLLTEVHAA
              660 670 680 690 700
SGSLPVLVVV VLVLLAVVLV TLAGIIVYRK ARSRILSRNV APKTTMGRSN
              710 720 730 740 750
PLFHQAASRV PAKGGAPAPS RGPQELVPTT HPGQPARHPA SSVALKRPPP
              760 770 780 790 800
APPVTVSSPP FPVPVYTRQA PKQVIKPTFA PPVPPVKPGA GAANPGPAEG
              810 820
AVGPFVALKP PIQRKQGAGA PTAP
(human ADAM8 consensus sequence where X35 = W or R)
SEQ ID NO: 13
MRGLGLWLLGAMMLPAIAPSRPWALMEQYEVVLP [symbol] RLPGPRVRRALPSHLGLHPERVS
YVLGATGHNFTLHLRKNRDLLGSGYTETYTAANGSEVTEQPRGQDHCFYQGHVEGYPD
SAASLSTCAGLRGFFQVGSDLHLIEPLDEGG [symbol] GGRHAVYQAEHLLQTAGTCGVSDDSLG
SLLGPRTAAVFRPRPGDSLPSRETRYVELYVVVDNAEFQMLGSEAAVRHRVLEVVNHVD
KLYQKLNFRVVLVGLEIWNSQDRFHVSPDPSVTLENLLTWQARQRTRRHLHDNVQLITG
VDFTGTTVGFARVSAMCSHSSGAVNQDHSKNPVGVACTMAHEMGHNLGMDHDENVQ
GCRCQERFEAGRCIMAGSIGSSFPRMFSDCSQAYLESFLERPQSVCLANAPDLSHLVGGPV
CGNLFVERGEQCDCGPPEDCRNRCCNSTTCQLAEGAQCAHGTCCQECKVKPAGELCRPK
KDMCDLEEFCDGRHPECPEDAFQENGTPCSGGYCYNGACPTLAQQCQAFWGPGGQAAE
ESCFSYDILPGCKASRYRADMCGVLQCKGGQQPLGRAICIVDVCHALTTEDGTAYEPVPE
GTRCGPEKVCWKGRCQDLHVYRSSNCSAQCHNHGVCNHKQECHCHAGWAPPHCAKLL
TEVHAASGSLPVFVVVVLVLLAVVLVTLAGIIVYRKARSRILSRNVAPKTTMGRSNPLFH
QAASRVPAKGGAPAPSRGPQELVPTTHPGQPARHPASSVALKRPPPAPPVTVSSPPFPVPV
YTRQAPKQVIKPTFAPPVPPVKPGAGAANPGPAEGAVGPKVALKPPIQRKQGAGAPTAP
(amino acids 17-196 of GENBANK® Accession with an N-terminal Met)
SEQ ID NO: 14
MIAPSRPWALMEQYEVVLP [symbol] RLPGPRVRRALPSHLGLHPERVSYVLGATGHNFTLHLRK
NRDLLGSGYTETYTAANGSEVTEQPRGQDHCFYQGHVEGYPDSAASLSTCAGLRGFFQV
GSDLHLIEPLDEGG [symbol] GGRHAVYQAEHLLQTAGTCGVSDDSLGSLLGPRTAAVFRPRPGDS
LPS
(embodiment of SEQ ID NO: 3)
SEQ ID NO: 15
MIAPSRPWALMEQYEVVLP [symbol] RLPGPRV [symbol] ALPSHLGLHPERVSYVLGATGHNFTLHLRK
NRDLLGSGYTETYTAANGSEVTEQPRGQDH [symbol] FYQGHVEGYPDSAASLST [symbol] AGLRGFFQV
GSDLHLIEPLDEGG [symbol] GGR [symbol] GT [symbol] GVSDDSLGSLLGPRTAAVFRPRPGD
SLPS
(embodiment of SEQ ID NO: 3)
SEQ ID NO: 16
MIAPSRPWALMEQYEVVLP [symbol] RLPGPRV [symbol] ALPSHLGLHPERVSYVLGATGHNFTLHLRK
NRDLLGSGYTETYTAANGSEVTEQPRGQDH [symbol] LYQGHVEGYPDSAASLST [symbol] AGLRGFFQV
GSDLHLIEPLDEGG [symbol] GGR [symbol] GT [symbol] GVSDDSLGSLLGPRTAAVFRPRPGD
SLPS
(embodiment of SEQ ID NO: 3)
SEQ ID NO: 17
MIAPSRPWALMEQYEVVLP [symbol] RLPGP [symbol] V [symbol] LPSHLGLHPERVSYVLGATGHNFTLHLR
KNRDLLGSGYTETYTAANGSEVTEQPRGQDH [symbol] FYQGHVEGYPDSAASLST [symbol] AGLRGFFQ
VGSDLHLIEPLDEGG [symbol] GGR [symbol] GT [symbol] GVSDDSLGSLLGPRTAAVFRPRPG
DSLPS
(2nd consensus sequence showing mutation sites for
the two furin cleavage sites and the cysteines)
SEQ ID NO: 18
MIAPSRPWALMEQYEVVLP [symbol] RLPGP [symbol] V [symbol] ALPSHLGLHPERVSYVLGATGHNFTLHL [symbol] N [symbol] DLLG
SGYTETYTAANGSEVTEQPRGQDH [symbol] FYQGHVEGYPDSAASLST [symbol] AGLRGFFQVGSDLHLIEPLDE
GG [symbol] GGRHAVYQAEHLLQTAGT [symbol] GVSDDSLGSLLGPRTAAVFRPRPGDSLPS
(3^{rd} consensus sequence showing mutation sites for
the two furin cleavage sites, the cysteines, and
autocatalytic sites)
SEQ ID NO: 19
MIAPSRPWALMEQYEVVLP [symbol] RLPGP [symbol] V [symbol] ALPSHLGLHPERVSYVLGAIGHNFTLHL [symbol] N [symbol] DLLG
SGYTETYTAANGSEVTEQPRGQDH [symbol] FYQGHVEGYPDSAASLST [symbol] AGLRGFFQVGSDLHLIEPLDE
GG [symbol] GGR [symbol] GT [symbol] GVSDDSLGSLLGPRTAAVFRPRPGDSLPS
(4th consensus sequence 3^{rd} consensus sequence
showing mutation sites for the two furin cleavage sites, the
cysteines, and autocatalytic sites)
SEQ ID NO: 20
MIAPSRPWALMEQYEVVLP [symbol] RLPGP [symbol] V [symbol] ALPSHLGLHPERVSYVLGAIGHN [symbol] FTLHL [symbol] N [symbol] DLL
GSGYTETYTAA [symbol] GSEVTEQPRGQDH [symbol] FYQGHVEGYPDSAASLST [symbol] AGLRGFFQVGSDLHLIEPLD
EGG [symbol] GGR [symbol] GT [symbol] GVSDDSLGSLLGPRTAAVFRPRPGDSLPS
(DNA sequence encoding modified ADAM8 prodomain optimized
for bacterial expression.)
SEQ ID NO: 21
ATGATTGCCCCCAGCCGGCCCTGGGCCCTCATGGAGCAGTATGAGGTCGTGTTGCCG
CGGCGTCTGCCAGGCCCCCGAGTCCGCCGAGCTCTGCCCTCCCACTTGGGCCTGCACC
CAGAGAGGGTGAGCTACGTCCTTGGGGCCACAGGGCACAACTTCACCCTCCACCTGC
GGAAGAACAGGGACCTGCTGGGTTCCGGCTACACAGAGACCTATACGGCTGCCAATG
GCTCCGAGGTGACGGAGCAGCCTCGCGGGCAGGACCACTGCTTATACCAGGGCCACG
TAGAGGGGTACCCGGACTCAGCCGCCAGCCTCAGCACCTGTGCCGGCCTCAGGGGTT
TCTTCCAGGTGGGGTCAGACCTGCACCTGATCGAGCCCCTGGATGAAGGTGGCGAGG
GCGGACGGCACGCCGTGTACCAGGCTGAGCACCTGCTGCAGACGGCCGGGACCTGCG
GGGTCAGCGACGACAGCCTGGGCAGCCTCCTGGGACCCCGGACGGCAGCCGTCTTCA
GGCCTCGGCCCGGGGACTCTCTGCCATCCGGATCTGGATCTCATCATCATCATCATCA
TTAA
(DNA sequence encoding modified ADAM8 prodomain optimized
for bacterial expression.)
SEQ ID NO: 22
ATGATTGCCCCCAGCCGGCCCTGGGCCCTCATGGAGCAGTATGAGGTCGTGTTGCCG
CGGCGTCTGCCAGGCCCCCGAGTCCGCGCTGCTCTGCCCTCCCACTTGGGCCTGCACC
CAGAGAGGGTGAGCTACGTCCTTGGGGCCACAGGGCACAACTTCACCCTCCACCTGC
GGAAGAACAGGGACCTGCTGGGTTCCGGCTACACAGAGACCTATACGGCTGCCAATG
GCTCCGAGGTGACGGAGCAGCCTCGCGGGCAGGACCACTCTTTCTACCAGGGCCACG
TAGAGGGGTACCCGGACTCAGCCGCCAGCCTCAGCACCTCTGCCGGCCTCAGGGGTT
TCTTCCAGGTGGGGTCAGACCTGCACCTGATCGAGCCCCTGGATGAAGGTGGCGAGG
GCGGACGGCACGCCGTGTACCAGGCTGAGCACCTGCTGCAGACGGCCGGGACCTCTG
GGGTCAGCGACGACAGCCTGGGCAGCCTCCTGGGACCCCGGACGGCAGCCGTCTTCA
GGCCTCGGCCCGGGGACTCTCTGCCATCCGGATCTGGATCTCATCATCATCATCATCA
TTAA
(DNA sequence encoding modified ADAM8 prodomain optimized
for bacterial expression.)
SEQ ID NO: 23
ATGGGCTCGTGTGGCTCCATTGCGCCGAGCCGTCCGTGGGCGCTGATGGAACAGTAT
GAAGTGGTGTTACCATGGCGGCTGCCCGGTCCGAAAGTGCGCAAAGCGCTGCCGTCG
CATCTGGGTCTGCACCCTGAGCGTGTGAGCTATGTGCTGGGCGCGACCGGCCATAAC
TTCACATTGCATCTTCGTAAAAATCGTGATTTACTGGGCTCCGGCTACACCGAAACCT
ATACGGCCGCTAATGGTAGCGAAGTGACCGAACAGCCGCGTGGCCAGGATCATAGCT
TTTACCAGGGCCATGTGGAAGGGTATCCTGATAGCGCGGCAAGCCTGAGCACATCGG
CTGGCCTGCGGGGCTTTTTTCAGGTGGGAAGCGACTTGCACCTGATTGAACCGCTGG
ATGAAGGCGGCGAAGGTGGGCGTCATGCGGTGTATCAGGCGGAACATCTGTTGCAGA
CAGCGGGCACAAGCGGCGTCAGCGACGATTCCCTTGGCAGCCTTCTGGGACCACGTA
CCGCGGCAGTTTTTCGTCCGCGTCCGGGCGACAGCCTGCCTAGCGGCTCTGGGAGCC
ATCACCACCATCATCACTAA
(DNA sequence encoding modified ADAM8 prodomain optimized
for bacterial expression.)
SEQ ID NO: 24
ATGATAGCGCCAAGCCGTCCCTGGGCGCTGATGGAACAGTATGAAGTGGTGCTGCCG
TGGCGTCTGCCAGGTCCACGTGTGCGTCGTGCGTTGCCGTCTCATCTGGGCCTGCACC
CTGAACGTGTGAGCTATGTGTTAGGCGCGACCGGCCATAATTTCACCCTGCATCTTCG
TAAAAATCGTGATCTGCTGGGCAGCGGCTATACCGAGACCTACACAGCGGCCAATGG
CAGCGAAGTGACCGAACAGCCGCGTGGCCAAGATCATTGCTTTTATCAGGGCCATGT
GGAAGGATACCCGGATTCAGCGGCGAGCCTGAGCACTTGCGCCGGTTTACGGGGCTT
TTTTCAGGTGGGGAGCGATTTGCATCTGATTGAACCGCTGGATGAAGGAGGGGAGGG
AGGCCGCCATGCGGTGTATCAAGCGGAGCATCTTTTACAAACCGCGGGAACCTGCGG
CGTGAGCGATGACAGCCTGGGGTCTTTGTTGGGACCACGTACCGCAGCGGTCTTTCGT
CCGCGTCCGGGCGATAGCTTACCGTCCGGCTCGGGGTCCCATCATCACCACCATCATT
GA
(DNA sequence encoding modified ADAM8 prodomain optimized
for bacterial expression.)
SEQ ID NO: 25
ATGATCGCGCCGTCTCGTCCGTGGGCGCTGATGGAACAGTATGAAGTGGTGCTGCCC
CGTCGTCTGCCGGGTCCCCGTGTGCGTGGCGCCCTGCCGAGCCATCTGGGCCTGCATC
CGGAACGTGTGAGCTATGTGCTGGGTGCGACCGGCCACAATTTTACCCTGCACCTGC
GTAAAAATCGTGATCTGCTGGGCTCAGGATATACCGAAACCTATACAGCGGCCAACG
GCTCAGAGGTGACCGAACAGCCGCGTGGCCAGGACCATAGCTTCTATCAGGGCCATG
TGGAAGGTTACCCCGACTCAGCAGCGAGCCTGAGCACAAGCGCGGGATTGCGTGGAT
TTTTTCAGGTGGGCAGCGATCTGCATCTGATTGAACCGCTGGATGAAGGGGGCGAAG
GAGGCCGTCATGCGGTGTATCAGGCGGAACACTTGCTGCAGACCGCGGGTACCTCTG
GCGTCAGCGATGACAGCCTGGGTAGCCTGTTGGGACCGCGTACCGCGGCAGTATTTC
GTCCGCGTCCGGGCGATAGCCTTCCGAGCGGTAGCTGCGGCTCCCATCATCACCACC
ATCATTAA
(DNA sequence encoding modified ADAM8 prodomain optimized
for bacterial expression.)
SEQ ID NO: 26
ATGATTGCCCCGAGCCGTCCGTGGGCGCTGATGGAACAGTATGAAGTGGTGCTGCCG
CGTCGTCTGCCGGGACCGCGTGTGCGTCGTGCGCTGCCGAGCCATCTGGGCCTGCATC
CGGAACGTGTGAGCTATGTGCTTGGCGCGACCGGCCATAATTTCACGTTGCATCTGCG
TAAAAATCGTGATCTGCTGGGCAGCGGCTATACCGAAACCTACACAGCTGCCAATGG
CAGCGAAGTGACCGAACAGCCGCGTGGCCAGGATCATTGCTTTTATCAGGGCCATGT
GGAGGGGTATCCAGATAGTGCCGCGAGCCTGAGCACCTGTGCGGGCTTGCGTGGCTT
TTTTCAGGTGGGGAGCGATCTGCATCTTATTGAACCGCTGGATGAAGGGGGCGAAGG
TGGCCGTCATGCCGTGTATCAAGCGGAACATTTACTGCAGACCGCGGGTACCTGCGG
CGTGAGCGATGATTCCCTGGGTAGCCTGTTGGGGCCTCGTACCGCTGCGGTGTTTCGT
CCGCGTCCGGGCGATAGTCTGCCTAGCGGTAGCGGGTCGCATCATCACCACCATCAC
TAA
(human ADAM8 prodomain)
SEQ ID NO: 27
ATTGCCCCCAGCCGGCCCTGGGCCCTCATGGAGCAGTATGAGGTCGTGTTGCCGTGG
CGTCTGCCAGGCCCCCGAGTCCGCCGAGCTCTGCCCTCCCACTTGGGCCTGCACCCAG
AGAGGGTGAGCTACGTCCTTGGGGCCACAGGGCACAACTTCACCCTCCACCTGCGGA
AGAACAGGGACCTGCTGGGCTCCGGCTACACAGAGACCTATACGGCTGCCAATGGCT
CCGAGGTGACGGAGCAGCCTCGCGGGCAGGACCACTGCTTCTACCAGGGCCACGTAG
AGGGGTACCCGGACTCAGCCGCCAGCCTCAGCACCTGTGCCGGCCTCAGGGGTTTCT
TCCAGGTGGGGTCAGACCTGCACCTGATCGAGCCCCTGGATGAAGGTGGCGAGGGCG
GACGGCACGCCGTGTACCAGGCTGAGCACCTGCTGCAGACGGCCGGGACCTGCGGG
GTCAGCGACGACAGCCTGGGCAGCCTCCTGGGACCCCGGACGGCAGCCGTCTTCAGG
CCTCGGCCCGGGGACTCTCTGCCATCC
```

1. A peptide comprising the amino acid sequence set forth in SEQ ID NO: 3 or 18-20, wherein relative to the amino acid sequence set forth in SEQ ID NO: 13, the peptide includes one or more amino acid substitutions and/or modifications at an amino acid position selected from the group consisting of amino acids 41, 43, 44, 67, 73, 74, 77, 91, 105, 124, 152-164, and 167, such that the peptide is auto-catalytically cleaved less, is less sensitive to furin cleavage, is less susceptible to oxidation and/or disulfide bond formation, less susceptible to degradation by ADAM8, or any combination thereof, as compared to a peptide without the one or more amino acid substitutions and/or modifications.

2. The peptide of claim 1, wherein the peptide comprises an amino acid sequence that is at least 90% identical to SEQ ID NO: 2, and further wherein as compared to SEQ ID NO: 13, the amino acid sequence comprises one or more amino acid substitutions and/or modifications selected from the group consisting of:
(a) a substitution of arginine 41 to another amino acid, optionally alanine, serine, glycine, or lysine;
(b) a substitution of arginine 43 to another amino acid, optionally alanine, serine, glycine, or lysine;
(c) a substitution of arginine 44 to another amino acid, optionally alanine, serine, glycine, or lysine;
(d) a substitution of arginine 73 to another amino acid, optionally alanine, serine, glycine, or lysine;
(e) a substitution of lysine 74 to another amino acid, optionally alanine, serine, or glycine;
(f) a substitution of arginine 76 to another amino acid, optionally alanine, serine, glycine, or lysine;
(g) a substitution of glutamine 156 to another amino acid, optionally serine, alanine, glycine or asparagine;
(h) a substitution of glutamate 158 to another amino acid, optionally serine, alanine, glycine or asparagine;
(i) a substitution of glutamine 162 to another amino acid, optionally serine, alanine, glycine or asparagine;
(j) a substitution of amino acids 152, 153, 154, 155, 157, 159, 150, 161 to another amino acid;
(k) a substitution of cysteine 105 to another amino acid, optionally serine, alanine, or glycine;
(1) a substitution of cysteine 124 to another amino acid, optionally serine, alanine, or glycine;
(m) a substitution of cysteine 167 to another amino acid, optionally serine, alanine, or glycine; and
(n) a chemical modification of one, two, or all three of cysteines 105, 124, and 167, or any combination thereof.

3. The peptide of claim 1, wherein as compared to SEQ ID NO: 13, the amino acid sequence has:
(i) a substitution at one, two, three, four, or all five of amino acids 41, 43, 44, 73, and 76, wherein each substitution is independently any amino acid other than the amino acid present in the corresponding position of SEQ ID NO: 2 at the corresponding position, optionally wherein each substitution is independently selected from the group consisting of alanine, serine, glycine, and lysine; or
(ii) a substitution at one or more of amino acids 156, 158 and 162, wherein each substitution is independently any amino acid other than the amino acid present in the corresponding position of SEQ ID NO: 2 at the corresponding position, optionally wherein each substitution is independently selected from the group consisting of asparagine, alanine, serine, and glycine; or
(iii) a substitution at one or more amino acids 152, 153, 154, 155, 157, 159, 150, 161, wherein each substitution is independently any amino acid other than the amino acid present in the corresponding position of SEQ ID NO: 2 at the corresponding position, optionally wherein each substitution is independently selected from the group consisting of any of the natural amino acids; or
(iv) a substitution at one, two, or all three of amino acids 105, 124, and 167, wherein each substitution is independently any amino acid other than cysteine, optionally wherein each substitution is independently selected from the group consisting of serine, alanine, and glycine;
(v) or any combination thereof.

4. The peptide of SEQ ID NO: 3, where X 35 of SEQ ID NO: 13 is arginine; arginine 41 is replaced with lysine; arginine 44 of SEQ ID NO: 13 is replaced with alanine, glycine, or lysine; and/or cysteines 105, 124, and 167 of SEQ ID NO: 13 are replaced with serines, or any combination thereof.

5. The peptide of claim 1, wherein one, two, or all three of cysteines 105, 124, and 167 of SEQ ID NO: 13 is chemically modified at its a sulfhydryl group, optionally wherein the sulfhydryl group(s) is/are chemically modified by addition of a maleimide ester, an α-halocarbonyl, a thiosulfonate, a disulfide derivative, or any combination thereof.

6. The peptide of claim 1, wherein with reference to SEQ ID NO: 13, the amino acid sequence comprises a substitution of one or more of arginine 41, 43, 44, 73, and/or 76 to an amino acid other than arginine, a substitution of lysine 74 to an amino acid other than lysine, a substitution of one or more of cysteines 105, 124, and 167 to an amino acid other than cysteine, optionally serine, or any combination thereof.

7. The peptide of claim 1, further comprising a pegylated cysteine added within 20 amino acids of the N-terminus, within 20 amino acids of the C-terminus, or both, optionally wherein one or both of the pegylated cysteines comprise a PEG group having a molecular weight of about 1 kiloDalton (kDa) to about 40 kDa.

8. The peptide of claim 1, wherein with reference to SEQ ID NO: 13, the amino acid sequence comprises a substitution of at least one of arginine 41, arginine 43, arginine 44, arginine 73, or arginine 76 to an amino acid other than arginine, and substitutions of cysteines 105, 124, and 167 to serine.

9. The peptide of claim 1, wherein with reference to SEQ ID NO: 13, the amino acid at position 105, 124, and/or 167 is cysteine and further wherein cysteine 105, 124, and/or 167 is pegylated, optionally wherein the pegylation comprises a PEG group having a molecular weight of about 1 kDa to about 40 kDa.

10. The peptide of claim 1, wherein with reference to SEQ ID NO: 13, one or more of cysteines 105, 124, and 167 comprises a chemical modification with a maleimide ester.

11. (canceled)

12. The peptide of claim 1, wherein with reference to SEQ ID NO: 13, one or more of cysteines 105, 124, and 167 comprises a chemical modification resulting from reacting the one or more cysteines with a disulfide.

13. (canceled)

14. The peptide of claim 1, wherein the peptide comprises an amino acid sequence having a percent identity of at least 87% to any one of SEQ ID NOs: 3-9 or 15-20, optionally wherein the percent identity is at least 95%.

15. The peptide of claim 14, wherein the peptide comprises an amino acid sequence having 100% percent identity to any one of SEQ ID NOs: 3-9 or 15-20 over its full length.

16. (canceled)

17. (canceled)

18. A composition comprising the peptide of claim 1, wherein the composition is formulated for administration to a subject or is a pharmaceutical composition formulated for administration to a human.

19. A fusion protein comprising the peptide of claim 1.

20. (canceled)

21. (canceled)

22. (canceled)

23. (canceled)

24. (canceled)

25. (canceled)

26. (canceled)

27. (canceled)

28. An isolated nucleic acid encoding the polypeptide of claim 1.

29. The isolated nucleic acid of claim 28, wherein the isolated nucleic acid comprises a nucleic acid sequence that is at least 97% identical to the nucleic acid sequence as set forth in SEQ ID NO: 21-26.

30. (canceled)

31. A vector comprising the isolated nucleic acid of claim 28.

32. A recombinant host cell comprising the vector of claim 31.

33. (canceled)

34. (canceled)

35. (canceled)

36. (canceled)

37. A method for modulating an ADAM8 biological activity in a eukaryotic cell which comprises contacting the cell with a genome modifying enzyme, whereby the genome modifying enzyme modifies a nucleic acid encoding an amino acid sequence set forth in SEQ ID NO: 13, such that the nucleic acid expresses a peptide that includes one or more amino acid substitutions and/or modifications at an amino acid position selected from the group consisting of amino acids 41, 43, 44, 67, 73, 74, 77, 91, 105, 124, 152-164, and 167, such that the expressed peptide is auto-catalytically cleaved less, is less sensitive to furin cleavage, is less susceptible to oxidation and/or disulfide bond formation, or any combination thereof, as compared to a peptide without the one or more amino acid substitutions and/or modifications.

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)
